(19) United States
(12) Patent Application Publication
(10) Pub. No.: US 2014/0248292 A1
(43) Pub. Date: Sep. 4, 2014

(54) COMPOSITIONS AND METHODS FOR TREATING CANCER

(71) Applicant: Oslo universitetssykehus HF, Oslo (NO)

(72) Inventors: Kjersti Flatmark, Blommenholm (NO); Yvonne Andersson, Nesoddtangen (NO); Oystein Fodstad, Oslo (NO)

(73) Assignee: Oslo universitetssykehus HF, Oslo (NO)

(21) Appl. No.: 14/196,604

(22) Filed: Mar. 4, 2014

Related U.S. Application Data
(60) Provisional application No. 61/772,155, filed on Mar. 4, 2013.

(51) Int. Cl. A61K 39/395; A61K 31/407; C07K 16/18

(52) U.S. Cl. CPC A61K 39/39558; C07K 16/18; A61K 31/407
USPC 424/174.1; 424/178.1

(57) ABSTRACT

The present invention relates to compositions and methods for treating cancer. In particular, the present invention relates to EpCAM-targeted immunotoxins and uses thereof in treating peritoneal cancers expressing EpCAM (e.g., colorectal, ovarian, and pancreatic cancer).

### CROSS-REFERENCE TO RELATED APPLICATIONS

The present application claims priority to pending U.S. Provisional Patent Application No. 61/772,155, filed Mar. 4, 2013, the contents of which are incorporated by reference in its entirety.

### FIELD OF THE INVENTION

The present invention relates to compositions and methods for treating cancer. In particular, the present invention relates to EpCAM-targeted immunotoxins and uses thereof in treating peritoneal cancers expressing EpCAM (e.g., colorectal, ovarian, and pancreatic cancer).

### BACKGROUND OF THE INVENTION

Mucinous peritoneal surface malignancies of intestinal origin encompass a range of clinical presentations, from the clinically benign manifestation pseudomyxoma peritonei (PMP) to aggressive mucinous peritoneal carcinomatosis from colorectal cancer. Histopathological presentation varies; with increasing cellular atypia, high epithelium to mucin ratio and signet ring cell differentiation being associated with an aggressive phenotype. The treatment is, regardless of histology, aimed at complete surgical removal of all visible tumor deposits followed by chemotherapy, typically some type of perioperative intraperitoneal (i.p.) chemotherapy, often supplemented with postoperative systemic chemotherapy. Prognosis is highly dependent on histopathological subtype, and particularly in peritoneal mucinous carcinomatosis (PMCA), the search for more efficacious chemotherapy regimens is warranted (Ronnett et al., Cancer 2001; 92:85-91; Rout et al., Dis Colon Rectum 2009; 52:1705-14; Yan et al., Br J Surg 2006; 93:1270-6.). Although the benefit of cytoreductive surgery is well documented in mucinous peritoneal surface malignancies, the optimal chemotherapeutic strategy has not been proven (Ronnett et al., supra; Rout et al., supra; Yan et al., supra; Verwaal et al., Ann Surg Oncol 2008; 15:2426-32). Several drug combinations and delivery strategies are currently being pursued, mostly in early clinical trials, while preclinical assessment of drug efficacy has been impeded by lack of appropriate experimental models.

Additional treatments for peritoneal and related cancers are needed.

### SUMMARY OF THE INVENTION

The present invention relates to compositions and methods for treating cancer. In particular, the present invention relates to EpCAM-targeted immunotoxins and uses thereof in treating peritoneal cancers expressing EpCAM (e.g., colorectal, ovarian, and pancreatic cancer).

Embodiments of the present invention provide improved methods of treating cancer (e.g., peritoneal surface malignancies of variant origin). In some embodiments, treatment methods comprise administering an immunotoxin targets EpCAM (e.g., MOC31PE) directly to the intraperitoneal cavity (e.g., via I.P injection) or systemically. In some embodiments, an additional agent (e.g., chemotherapeutic agent (e.g., mitomycin C)) is administered in combination with the immunotoxin.

For example, in some embodiments, the present invention provides a method of treating intraperitoneal cancers (e.g., colorectal cancer, pancreatic cancer, ovarian cancer or a mucinous peritoneal surface malignancy), comprising: administering an immunotin (e.g., comprising an antibody that binds to EpCAM conjugated to a cytotoxin), alone or in combination with a chemotherapeutic agent (e.g., mitomycin C) to a subject diagnosed with a cancer that expresses EpCAM (e.g., ovarian cancer, pancreatic cancer, colorectal cancer, or mucinous peritoneal surface malignancy). In some embodiments, the administering is via intraperitoneal injection. In some embodiments, the administering reduces symptoms, growth, or presence of the cancer. In some embodiments, the mucinous peritoneal surface malignancy is pseudomyxoma peritonei, colorectal, ovarian, or pancreatic cancers that have metastasized to the peritoneal cavity, or peritoneal carcinomatosis. In some embodiments, the subject has undergone cytoreductive surgery. In some embodiments, the antibody is MOC31PE (e.g., MOC31 linked to pseudomonas exotoxin A). Exemplary dosages of the therapies include, but are not limited to, 5 to 150 μg/kg of MOC31PE and 1 to 5 mg/kg of mitomycin C. In some embodiments, the immunotoxin and chemotherapeutic agent are administered concurrently or successively (e.g., in the same solution or different solutions). In some embodiments, the immunotoxin and the chemotherapeutic drug are both administered intraperitoneally. In some embodiments, the immunotoxin is administered intraperitoneally, and the chemotherapeutic drug by another systemic route.

Embodiments of the present invention comprise a method of treating a cancer that expresses EPCAM, comprising: a) assaying a sample from a subject diagnosed with cancer for the presence of EpCAM expression; and b) administering an immunotoxin comprising an antibody that binds to EpCAM (e.g. alone or in combination with an additional chemotherapeutic agent) when the cancer expresses EpCAM.

In some embodiments, the present invention provides a composition comprising a combination of a chemotherapeutic drug (e.g., mitomycin C) and an immunotoxin that targets EpCAM. In some embodiments, the composition is a solution formulated for I. P. injection. In some embodiments, the immunotoxin and the chemotherapeutic agent are formulated in the same solution. In some embodiments, the chemotherapeutic agent and the immunotoxin are formulated in separate solutions or vials and are packaged together in the same shipping or storage container.

Additional embodiments are described herein.

### DESCRIPTION OF THE DRAWINGS

FIG. 1 shows efficacy of intraperitoneal (i.p.) mitomycin C (MMC) and oxaliplatin (OXA) in five orthotopic models of mucinous peritoneal surface malignancies. A Mean growth indices calculated for the PMCA-2 model showing the effect of administrating single i.p. doses of MMC, OXA and vehicle, (on day 1 or day 6 after tumor implantation). B Mean growth indices calculated for the PMP-1, PMP-2, PMCA-1 and PMCA-3 models after single i.p. doses of vehicle, MMC 2.5 mg/kg, MMC 5 mg/kg and OXA 10 mg/kg (on day 1 after tumor implantation).

FIG. 2 shows expression of EpCAM as assessed by immunohistochemistry; PMP-2 (A), PMCA-2 (B) models, negative control (C) and western immunoblot analysis of lysates from all five xenografts (D).

FIG. 3 shows mean growth indices for the PMCA-2, PMP-2 and PMCA-3 models to demonstrate the effects of administrating single intraperitoneal doses of vehicle, mitomycin C (MMC), MOC31PE and combinations of the drugs. Error bars indicate standard deviation. APMCA-2; B PMP-2; C PMCA-3

FIG. 4 shows ex vivo short-term cultures of mucinous tumor tissue from the PMCA-2 and PMP-2 animal models and of two samples of mucinous tumor tissue taken directly from the operating theatre. A Cell viability was assessed 24 h after addition of MOC31PE (1000 ng/ml) using the MTS assay and is expressed as percentage of the value obtained in vehicle treated cells. B and C Protein synthesis inhibition was determined by measuring ³H-leucine incorporation after treatment with MOC31PE or vehicle in PMCA-2 (B) and PMP-2 (C) and is expressed as a percentage of the value obtained in vehicle treated cells. D Western blot analysis was performed on protein lysates after exposure to MOC31PE or vehicle for 24 h.

FIG. 5 shows the inhibitory effect of MOC31PE immunotoxin on protein synthesis and cell viability. B76 cells were seeded in 48-well plates (a) and the next day the medium were changed and added IT and/or CsA. The lower graph (b) is one representative experiment of three independent experiments with treatment for 48 h. Average±SD for each treatment tested in triplicate is shown.

FIG. 6 shows increased membrane leakage followed in an IncuCyte live-imaging device. 76 cells were seeded in 96-well plates, IT and/or CsA were added and the fluorescence measured every second hour for 48 h (a). The cytotoxic index (b) is the ratio of fluorescent objects before and after lysis of the cells by adding Triton X-100.

FIG. 7 shows the inhibitory effect of IT+/−CsA in a scratch-wound healing assay followed in an IncuCyte. B76 cells were seeded in 96-well plates (Essen image lock) and scratch wounds made simultaneously in all wells using the wound maker tool. Relative wound density (RWD), defined as the ratio of the cell density in the wound over the cell density outside the wound, was measured every second hour for up to 22 h (a). Average RWD after 22 h in five independent experiments is summarized in (b).

FIG. 8 shows mechanisms for NR4A3 protein involvement during IT-induced cell death. B76 cells were seeded in 25 cm² flasks and treated for 24 h with IT (10 ng/ml), CsA (2 μM) or the combination when cells were 70% confluent. Cell lysates were prepared and 15 μg total protein added each SDS-PAGE lane (a). The apparent molecular weight for the main band is 60 kDa and around 55 kDa for the lower band. In (b) cells were seeded in 75 cm² flasks and treated as in (a).

FIG. 9 shows cell surface expression of EpCAM detected using magnetic beads coated with the MOC31 antibody. Upper panel show very good binding and thus high expression of the antigen EpCAM whereas no binding was seen with control beads.

FIG. 10 shows pictures of B76 cells taken immediately after scratching confluent cell layers (0 h) and after incubating wells with media containing MOC31PE (10 ng/ml) or CsA+MOC31PE for 24 hours in the scratch assay.

FIG. 11 shows protein synthesis in HOC-7 ovarian cancer cells after 24 h incubation with MOC31PE.

FIG. 12 shows the effect of MOC31PE on HOC-7 ovarian cancer cell viability measured using the MTS-assay.

FIG. 13 shows gene expression of selected genes in HOC-7 ovarian cancer cells tested in qPCR with Taqman probes.

FIG. 14 shows that MOC31PE efficiently inhibits the cell viability of B76 and 2774 ovarian cancer cells.

FIG. 15 shows that MOC31PE given in the abdominal cavity (i.p.) effectively inhibits B76 ovarian peritoneal carcinomatosis in experimental animal model.

FIG. 16 shows that MOC31PE given in the abdominal cavity (i.p.) effectively inhibits 2774 ovarian peritoneal carcinomatosis in experimental animal model.

FIG. 17 shows inhibitory effect of MOC31PE on short-term cultures of fresh human ovarian tumor tissue taken directly from the operating theatre.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and/or molecular screening tests.

As used herein, the term “initial diagnosis” refers to a test result of initial cancer diagnosis that reveals the presence or absence of cancerous cells (e.g., using a biopsy and histology).

As used herein, the term “characterizing cancer in subject” refers to the identification of one or more properties of a cancer sample in a subject, including but not limited to, the presence of benign, pre-cancerous or cancerous tissue and the stage of the cancer.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

Staging of cancer can also be based on the revised criteria of TNM staging by the American Joint Committee for Cancer (AJCC) published in 1988. Staging is the process of describing the extent to which cancer has spread from the site of its origin. It is used to assess a patient's prognosis and to determine the choice of therapy. The stage of a cancer is determined by the size and location in the body of the primary tumor, and whether it has spread to other areas of the body. Staging involves using the letters T, N and M to assess tumors by the size of the primary tumor (T); the degree to which regional lymph nodes (N) are involved; and the absence or presence of distant metastases (M)—cancer that has spread from the original (primary) tumor to distant organs or distant lymph nodes. Each of these categories is further classified with a number 1 through 4 to give the total stage. Once the T, N and M are determined, a “stage” of I, II, III or IV is assigned. Stage I cancers are small, localized and usually curable. Stage II and III cancers typically are locally advanced and/or have spread to local lymph nodes. Stage IV cancers usually are metastatic (have spread to distant parts of the body) and generally are considered inoperable.

As used herein, the term “characterizing tissue in a subject” refers to the identification of one or more properties of a tissue sample (e.g., including but not limited to, the presence of cancerous tissue, the presence of pre-cancerous tissue that is likely to become cancerous, and the presence of cancerous tissue that is likely to metastasize).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present invention.

As used herein, the term “co-administration” refers to the administration of at least two agent(s) (e.g., a immunotoxin that targets EpCAM and a chemotherapeutic agent) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In other embodiments, suitable monoclonal antibodies, including recombinant chimeric monoclonal antibodies and chimeric monoclonal antibody fusion proteins are prepared as described herein.

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

In some embodiments, monoclonal antibodies are generated using the ABL-MYC method (See e.g., U.S. Pat. Nos. 5,705,150 and 5,244,656, each of which is herein incorporated by reference) (Neoclone, Madison, Wis.). ABL-MYC is a recombinant retrovirus that constitutively expresses v-abl and c-myc oncogenes. When used to infect antigen-activated splenocytes, this retroviral system rapidly induces antigen-specific plasmacytomas. ABL-MYC targets antigen-stimulated (Ag-stimulated) B-cells for transformation.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

### DETAILED DESCRIPTION OF THE INVENTION

The present invention relates to compositions and methods for treating cancer. In particular, the present invention relates to EpCAM-targeted immunotoxins and uses thereof in treating peritoneal cancers expressing EpCAM (e.g., colorectal, ovarian, and pancreatic cancer).

Cytoreductive surgery and intraperitoneal (i.p.) chemotherapy constitute a curative treatment option in mucinous peritoneal surface malignancies of intestinal origin, but treatment outcome is highly variable and the search for novel therapies is warranted. Immunotoxins are attractive candidates for targeted therapy in the peritoneal cavity because of direct cytotoxicity, distinct mechanisms of action and tumor cell selectivity. The MOC31PE immunotoxin targets the tumor-associated adhesion protein EpCAM (Epithelial Cell Adhesion Molecule), and has been administered safely in early clinical trials.

Experiments conducted during the course of development of embodiments of the present invention demonstrated the efficacy of i.p. administration of MOC31PE alone and together with mitomycin C (MMC) in animal models of human mucinous peritoneal surface malignancies. MOC31PE and MMC were given as single i.p. injections alone and in combination. In the PMCA-2 model, moderate growth inhibition was obtained with both drugs, while the combination resulted in at least additive effects; whereas the PMP-2 model was highly sensitive to both drugs separately and in combination and intermediate sensitivity was found for the PMCA-3 model. Furthermore, results from ex vivo experiments on freshly obtained mucinous tumor tissue from animals and patients indicated that inhibition of protein synthesis and induction of apoptosis was present. The present results indicate that adding MOC31PE to MMC-based i.p. chemotherapy provides an improved treatment for EpCAM-expressing peritoneal surface malignancies.

### I. Immunotoxins

A. Immunoglobulins

Embodiments of the present invention provide immunotoxins comprising immunoglobulins directed to EpCAM, alone or in combination with a chemotherapeutic agent. In some embodiments, the immunoglobulin is MOC31PE. The MOC31PE immunotoxin is composed of the MOC31 monoclonal antibody targeting the tumor-associated antigen EpCAM, covalently linked to pseudomonas exotoxin A (PE). The antibody targets the immunotoxin to EpCAM-expressing cells, and when internalized, the toxin effector moiety triggers cell death by catalytic inactivation of vital processes, such as protein synthesis, and by directly inducing apoptosis. The present disclosure is not limited to MOC31PE. Additional immunotoxins and immunoglobulins are specifically contemplated

Immunoglobulins (antibodies) are proteins generated by the immune system to provide a specific molecule capable of complexing with an invading molecule commonly referred to as an antigen. Natural antibodies have two identical antigen-binding sites, both of which are specific to a particular antigen. The antibody molecule recognizes the antigen by complexing its antigen-binding sites with areas of the antigen termed epitopes. The epitopes fit into the conformational architecture of the antigen-binding sites of the antibody, enabling the antibody to bind to the antigen.

The immunoglobulin molecule is composed of two identical heavy and two identical light polypeptide chains, held together by interchain disulfide bonds. Each individual light and heavy chain folds into regions of about 110 amino acids, assuming a conserved three-dimensional conformation. The light chain comprises one variable region (termed V_{L}) and one constant region (C_{L}), while the heavy chain comprises one variable region (V_{H}) and three constant regions (C_{H}1, C_{H}2 and C_{H}3). Pairs of regions associate to form discrete structures. In particular, the light and heavy chain variable regions, V_{L} and V_{H}, associate to form an “F_{V}” area that contains the antigen-binding site.

The variable regions of both heavy and light chains show considerable variability in structure and amino acid composition from one antibody molecule to another, whereas the constant regions show little variability. Each antibody recognizes and binds an antigen through the binding site defined by the association of the heavy and light chain, variable regions into an F_{V} area. The light-chain variable region V_{L} and the heavy-chain variable region V_{H} of a particular antibody molecule have specific amino acid sequences that allow the antigen-binding site to assume a conformation that binds to the antigen epitope recognized by that particular antibody.

Within the variable regions are found regions in which the amino acid sequence is extremely variable from one antibody to another. Three of these so-called “hypervariable” regions or “complementarity-determining regions” (CDR's) are found in each of the light and heavy chains. The three CDRs from a light chain and the three CDRs from a corresponding heavy chain form the antigen-binding site.

Cleavage of naturally occurring antibody molecules with the proteolytic enzyme papain generates fragments that retain their antigen-binding site. These fragments, commonly known as Fab's (for Fragment, antigen binding site) are composed of the C_{L}, V_{L}, C_{H}1 and V_{H} regions of the antibody. In the Fab the light chain and the fragment of the heavy chain are covalently linked by a disulfide linkage.

Monoclonal antibodies against target antigens (e.g., a cell surface protein, such as receptors) are produced by a variety of techniques including conventional monoclonal antibody methodologies such as the somatic cell hybridization techniques of Kohler and Milstein, Nature, 256:495 (1975). Although in some embodiments, somatic cell hybridization procedures are preferred, other techniques for producing monoclonal antibodies are contemplated as well (e.g., viral or oncogenic transformation of B lymphocytes).

The preferred animal system for preparing hybridomas is the murine system. Hybridoma production in the mouse is a well-established procedure Immunization protocols and techniques for isolation of immunized splenocytes for fusion are known in the art. Fusion partners (e.g., murine myeloma cells) and fusion procedures are also known.

Human monoclonal antibodies (mAbs) directed against human proteins can be generated using transgenic mice carrying the complete human immune system rather than-the mouse system. Splenocytes from the transgenic mice are immunized with the antigen of interest, which are used to produce hybridomas that secrete human mAbs with specific affinities for epitopes from a human protein. (See e.g., Wood et al., WO 91/00906, Kucherlapati et al., WO 91/10741; Lonberg et al., WO 92/03918; Kay et al., WO 92/03917 [each of which is herein incorporated by reference in its entirety]; N. Lonberg et al., Nature, 368:856-859 [1994]; L. L. Green et al., Nature Genet., 7:13-21 [1994]; S. L. Morrison et al., Proc. Nat. Acad. Sci. USA, 81:6851-6855 [1994]; Bruggeman et al., Immunol., 7:33-40 [1993]; Tuaillon et al., Proc. Nat. Acad. Sci. USA, 90:3720-3724 [1993]; and Bruggernan et al. Eur. J. Immunol., 21:1323-1326 [1991]).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies. (See e.g., Sastry et al., Proc. Nat. Acad. Sci. USA, 86:5728 [1989]; Huse et al., Science, 246:1275 [1989]; and Orlandi et al., Proc. Nat. Acad. Sci. USA, 86:3833 [1989]). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and the PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies. (See e.g., Larrick et al., Biotechniques, 11:152-156 [1991]). A similar strategy can also been used to amplify human heavy and light chain variable regions from human antibodies (See e.g., Larrick et al., Methods: Companion to Methods in Enzymology, 2:106-110 [1991]).

In one embodiment, RNA is isolated from B lymphocytes, for example, peripheral blood cells, bone marrow, or spleen preparations, using standard protocols (e.g., U.S. Pat. No. 4,683,292 [incorporated herein by reference in its entirety]; Orlandi, et al., Proc. Nat. Acad. Sci. USA, 86:3833-3837 [1989]; Sastry et al., Proc. Nat. Acad. Sci. USA, 86:5728-5732 [1989]; and Huse et al., Science, 246:1275 [1989]). First strand cDNA is synthesized using primers specific for the constant region of the heavy chain(s) and each of the κ and λ light chains, as well as primers for the signal sequence. Using variable region PCR primers, the variable regions of both heavy and light chains are amplified, each alone or in combination, and ligated into appropriate vectors for further manipulation ingenerating the display packages. Oligonucleotide primers useful in amplification protocols may be unique or degenerate or incorporate inosine at degenerate positions. Restriction endonuclease recognition sequences may also be incorporated into the primers to allow for the cloning of the amplified fragment into a vector in a predetermined reading frame for expression.

The V-gene library cloned from the immunization-derived antibody repertoire can be expressed by a population of display packages, preferably derived from filamentous phage, to form an antibody display library. Ideally, the display package comprises a system that allows the sampling of very large variegated antibody display libraries, rapid sorting after each affinity separation round, and easy isolation of the antibody gene from purified display packages. In addition to commercially available kits for generating phage display libraries, examples of methods and reagents particularly amenable for use in generating a variegated antibody display library can be found in, for example, U.S. Pat. No. 5,223,409; WO 92/18619; WO 91/17271; WO 92/20791; WO 92/15679; WO 93/01288; WO 92/01047; WO 92/09690; WO 90/02809 [each of which is herein incorporated by reference in its entirety]; Fuchs et al., Biol. Technology, 9:1370-1372 [1991]; Hay et al., Hum. Antibod. Hybridomas, 3:81-85 [1992]; Huse et al., Science, 46:1275-1281 [1989]; Hawkins et al., J. Mol. Biol., 226:889-896 [1992]; Clackson et al., Nature, 352:624-628 [1991]; Gram et al., Proc. Nat. Acad. Sci. USA, 89:3576-3580 [1992]; Garrad et al., Bio/Technolog, 2:1373-1377 [1991]; Hoogenboom et al., Nuc. Acid Res., 19:4133-4137 [1991]; and Barbas et al., Proc. Nat. Acad. Sci. USA, 88:7978 [1991]. In certain embodiments, the V region domains of heavy and light chains can be expressed on the same polypeptide, joined by a flexible linker to form a single-chain Fv fragment, and the scFV gene subsequently cloned into the desired expression vector or phage genome.

As generally described in McCafferty et al., Nature, 348:552-554 (1990), complete V_{H} and V_{L} domains of an antibody, joined by a flexible linker (e.g., (Gly₄-Ser)₃) can be used to produce a single chain antibody which can render the display package separable based on antigen affinity. Isolated scFV antibodies immunoreactive with the antigen can subsequently be formulated into a pharmaceutical preparation for use in the subject method.

Once displayed on the surface of a display package (e.g., filamentous phage), the antibody library is screened with the target antigen, or peptide fragment thereof, to identify and isolate packages that express an antibody having specificity for the target antigen. Nucleic acid encoding the selected antibody can be recovered from the display package (e.g., from the phage genome) and subcloned into other expression vectors by standard recombinant DNA techniques.

Specific antibody molecules with high affinities for a surface protein can be made according to methods known to those in the art, e.g., methods involving screening of libraries U.S. Pat. No. 5,233,409 and U.S. Pat. No. 5,403,484 (both incorporated herein by reference in their entireties). Further, the methods of these libraries can be used in screens to obtain binding determinants that are mimetics of the structural determinants of antibodies.

In particular, the Fv binding surface of a particular antibody molecule interacts with its target ligand according to principles of protein-protein interactions, hence sequence data for V_{H} and V_{L} (the latter of which may be of the κ or λ chain type) is the basis for protein engineering techniques known to those with skill in the art. Details of the protein surface that comprises the binding determinants can be obtained from antibody sequence in formation, by a modeling procedure using previously determined three-dimensional structures from other antibodies obtained from NMR studies or crystallographic data.

In one embodiment, a variegated peptide library is expressed by a population of display packages to form a peptide display library. Ideally, the display package comprises a system that allows the sampling of very large variegated peptide display libraries, rapid sorting after each affinity separation round, and easy isolation of the peptide-encoding gene from purified display packages. Peptide display libraries can be in, e.g., prokaryotic organisms and viruses, which can be amplified quickly, are relatively easy to manipulate, and which allows the creation of large number of clones. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages. Phage display libraries are known in the art.

Other techniques include affinity chromatography with an appropriate “receptor,” e.g., a target antigen, followed by identification of the isolated binding agents or ligands by conventional techniques (e.g., mass spectrometry and NMR). Preferably, the soluble receptor is conjugated to a label (e.g., fluorophores, colorimetric enzymes, radioisotopes, or luminescent compounds) that can be detected to indicate ligand binding. Alternatively, immobilized compounds can be selectively released and allowed to diffuse through a membrane to interact with a receptor.

Combinatorial libraries of compounds can also be synthesized with “tags” to encode the identity of each member of the library. (See e.g., W. C. Still et al., WO 94/08051 incorporated herein by reference in its entirety). In general, this method features the use of inert but readily detectable tags that are attached to the solid support or to the compounds. When an active compound is detected, the identity of the compound is determined by identification of the unique accompanying tag. This tagging method permits the synthesis of large libraries of compounds that can be identified at very low levels among to total set of all compounds in the library.

The term modified antibody is also intended to include antibodies, such as monoclonal antibodies, chimeric antibodies, and humanized antibodies which have been modified by, for example, deleting, adding, or substituting portions of the antibody. For example, an antibody can be modified by deleting the hinge region, thus generating a monovalent antibody. Any modification is within the scope of the invention so long as the antibody has at least one antigen binding region specific.

Chimeric mouse-human monoclonal antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (See e.g., Robinson et al., PCT/US86/02269; European Patent Application 184,187; European Patent Application 171,496; European Patent Application 173,494; WO 86/01533; U.S. Pat. No. 4,816,567; European Patent Application 125,023 [each of which is herein incorporated by reference in its entirety]; Better et al., Science, 240:1041-1043 [1988]; Liu et al., Proc. Nat. Acad. Sci. USA, 84:3439-3443 [1987]; Liu et al., J. Immunol., 139:3521-3526 [1987]; Sun et al., Proc. Nat. Acad. Sci. USA, 84:214-218 [1987]; Nishimura et al., Canc. Res., 47:999-1005 [1987]; Wood et al., Nature, 314:446-449 [1985]; and Shaw et al., J. Natl. Cancer Inst., 80:1553-1559 [1988]).

The chimeric antibody can be further humanized by replacing sequences of the Fv variable region that are not directly involved in antigen binding with equivalent sequences from human Fv variable regions. General reviews of humanized chimeric antibodies are provided by S. L. Morrison, Science, 229:1202-1207 (1985) and by Oi et al., Bio. Techniques, 4:214 (1986). Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPII_{b}IIIₐ antibody producing hybridoma. The recombinant DNA encoding the chimeric antibody, or fragment thereof, can then be cloned into an appropriate expression vector.

Suitable humanized antibodies can alternatively be produced by CDR substitution (e.g., U.S. Pat. No. 5,225,539 (incorporated herein by reference in its entirety); Jones et al., Nature, 321:552-525 [1986]; Verhoeyan et al., Science, 239:1534 [1988]; and Beidler et al., J. Immunol., 141:4053 [1988]). All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method that is capable of replacing at least a portion of a CDR of a human antibody with a CDR derived from a non-human antibody. The human CDRs may be replaced with non-human CDRs; using oligonucleotide site-directed mutagenesis.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

In some embodiments, the monoclonal antibody is a murine antibody or a fragment thereof. In other preferred embodiments, the monoclonal antibody is a bovine antibody or a fragment thereof. For example, the murine antibody can be produced by a hybridoma that includes a B cell obtained from a transgenic mouse having a genome comprising a heavy chain transgene and a light chain transgene fused to an immortalized cell. The antibodies can be of various isotypes, including, but not limited to: IgG (e.g., IgG1, IgG2, IgG2a, IgG2b, IgG2c, IgG3, IgG4); IgM; IgA1; IgA2; IgA_{sec}; IgD; and IgE. In some preferred embodiments, the antibody is an IgG isotype. In other preferred embodiments, the antibody is an IgM isotype. The antibodies can be full-length (e.g., an IgG1, IgG2, IgG3, or IgG4 antibody) or can include only an antigen-binding portion (e.g., a Fab, F(ab′)₂, Fv or a single chain Fv fragment).

In preferred embodiments, the immunoglobulin is a recombinant antibody (e.g., a chimeric or a humanized antibody), a subunit, or an antigen binding fragment thereof (e.g., has a variable region, or at least a complementarity determining region (CDR)).

In some embodiments, the immunoglobulin is monovalent (e.g., includes one pair of heavy and light chains, or antigen binding portions thereof). In other embodiments, the immunoglobulin is a divalent (e.g., includes two pairs of heavy and light chains, or antigen binding portions thereof).

In still other embodiments, the recombinant antibodies comprise an immunoglobulin having only heavy chains such as the HCAbs found in certain Camelidae (e.g., camels, dromedaries, and llamas) species, spotted ratfish, and nurse shark. While the present invention is not limited to any particular mechanisms, the present invention contemplates that there are differences between conventional antibodies and HCAbs in both the V_{H} and C_{H} regions. For instance, as reported by Muyldermans et al. and Nguyen et al., the sequences of HCAbs variable domains (V_{H}H) differ significantly from those of conventional antibodies (V_{H}). (S. Muyldermans et al., Protein, Eng., 7:1129-1135 [1994]; V. K. Nguyen et al., J. Mol. Biol., 275:413-418 [1998]; and V. K. Nguyen et al., Immunogenetics DOI 10.1007/s00251-002-0433-0 [2002]). Additionally, HCAbs lack the first domain of the constant region (C_{H}); the matured V_{H}H-DJ is directly joined to the hinge region. Separate sets of V and C genes encode conventional antibodies and HCAbs, however, conventional antibodies and HCAbs have some common D genes and appear to have identical J_{H} regions. (V. K. Nguyen et al., EMBO J., 19:921-930 [2000]; and V. K. Nguyen et al., Adv. Immunol., 79:261-296 [2001]).

In some embodiments, a system of hybridoma-like antibody preparation, developed by Neoclone (Madison, Wis.), is used in the production of monoclonal antibodies. Splenocytes from immunized mice are immortalized using a retrovector-mediated introduction of the abl-myc genes. On reintroduction into recipient mice one dominant immortalized B cell clone (plasmacytoma) outgrows all others and produces a monoclonal antibody in the ascitic fluid. The B cell clone can be harvested with the ascitic fluid that contains high concentration of monoclonal antibody. This process can be completed in 8-10 weeks.

B. Toxins

In some embodiments, antibodies targeting EpCAM are conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeting EpCAM. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

C. Pharmaceutical Formulations

Where clinical applications are contemplated, in some embodiments of the present invention, the immunotoxins are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight immunotoxin composition formulation may be administered using one or more of the routes described herein.

In some embodiments, the immunotoxin compositions are used in conjunction with appropriate salts and buffers to render delivery of the compositions in a stable manner to allow for uptake by target cells. Buffers also are employed when the compositions are introduced into a patient. Aqueous compositions comprise an effective amount of composition dispersed in a pharmaceutically acceptable carrier or aqueous medium. Such compositions also are referred to as inocula.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The compositions may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, compositions are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer.

D. Dosage

“Treating” within the context of the instant invention, means an alleviation, in whole or in part, of symptoms associated with a disorder or disease, or slowing, inhibiting or halting of further progression or worsening of those symptoms, or prevention or prophylaxis of the disease or disorder in a subject at risk for developing the disease or disorder. Thus, e.g., treating cancer may include inhibiting or preventing the metastasis of the cancer, a reduction in the speed and/or number of the metastasis, a reduction in tumor volume of the metastasized cancer, a complete or partial remission of the metastasized cancer or any other therapeutic benefit. As used herein, a “therapeutically effective amount” of a compound of the invention refers to an amount of the compound that alleviates, in whole or in part, symptoms associated with a disorder or disease, or slows, inhibits or halts further progression or worsening of those symptoms, or prevents or provides prophylaxis for the disease or disorder in a subject at risk for developing the disease or disorder.

A subject is any animal that can benefit from the administration of a compound as described herein. In some embodiments, the subject is a mammal, for example, a human, a primate, a dog, a cat, a horse, a cow, a pig, a rodent, such as for example a rat or mouse. Typically, the subject is a human.

A therapeutically effective amount of a compound as described herein used in the present invention may vary depending upon the route of administration and dosage form. Effective amounts of invention compounds typically fall in the range of about 0.001 up to 100 mg/kg/day, and more typically in the range of about 0.05 up to 10 mg/kg/day. Typically, the compound or compounds used in the instant invention are selected to provide a formulation that exhibits a high therapeutic index. The therapeutic index is the dose ratio between toxic and therapeutic effects which can be expressed as the ratio between LD50 and ED50. The LD50 is the dose lethal to 50% of the population and the ED50 is the dose therapeutically effective in 50% of the population. The LD50 and ED50 are determined by standard pharmaceutical procedures in animal cell cultures or experimental animals.

E. Routes of Administration

The instant invention also provides for pharmaceutical compositions and medicaments which may be prepared by combining one or more compounds described herein, pharmaceutically acceptable salts thereof, stereoisomers thereof, tautomers thereof, or solvates thereof, with pharmaceutically acceptable carriers, excipients, binders, diluents or the like to inhibit or treat primary and/or metastatic prostate cancers. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, or via implanted reservoir. Parenteral or systemic administration includes, but is not limited to, subcutaneous, intravenous, intraperitoneal, and intramuscular injections. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or antioxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, com oil and olive oil. Suspension preparations may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Compounds of the invention may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of inventive compounds by inhalation.

Ordinarily, an aqueous aerosol is made by formulating an aqueous solution or suspension of the compound together with conventional pharmaceutically acceptable carriers and stabilizers. The carriers and stabilizers vary with the requirements of the particular compound, but typically include nonionic surfactants (TWEENs, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver compounds of the invention.

Aerosols containing compounds for use according to the present invention are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

Delivery of aerosols of the present invention using sonic nebulizers is advantageous because nebulizers minimize exposure of the agent to shear, which can result in degradation of the compound.

For nasal administration, the pharmaceutical formulations and medicaments may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the compounds maybe formulated in oily solutions or as a gel. For administration of nasal aerosol, any suitable propellant may be used including compressed air, nitrogen, carbon dioxide, or a hydrocarbon based low boiling solvent.

Dosage forms for the topical (including buccal and sublingual) or transdermal administration of compounds of the invention include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the invention to the body. Such dosage forms can be made by dissolving or dispersing the agent in the proper medium. Absorption enhancers can also be used to increase the flux of the inventive compound across the skin. The rate of such flux can be controlled by either providing a rate controlling membrane or dispersing the compound in a polymer matrix or gel.

Besides those representative dosage forms described above, pharmaceutically acceptable excipients and carriers are generally known to those skilled in the art and are thus included in the instant invention. Such excipients and carriers are described, for example, in

“Remingtons Pharmaceutical Sciences” Mack Pub. Co., New Jersey (1991), which is incorporated herein by reference.

The formulations of the invention may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

### II. Methods of Treating Epithelial Cancer

In some embodiments of the present invention, methods and compositions are provided for the treatment of tumors and cancers than express EpCAM (e.g., ovarian or peritoneal cancers). In some embodiments, the cancer is a mucinous peritoneal surface malignancy (e.g., pseudomyxoma peritonei or peritoneal carcinomatosis).

It is contemplated that such therapy can be employed in the treatment of any cancer for which a specific signature has been identified or which can be targeted (e.g. EpCAM expression). EpCAM expressing cancers can be identified using any suitable method (e.g., those disclosed herein).

Cell proliferative disorders, or cancers, contemplated to be treatable with the methods of the present invention include human sarcomas and carcinomas, including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia and acute myelocytic leukemia (myeloblastic, promyelocytic, myelomonocytic, monocytic and erythroleukemia), chronic leukemia (chronic myelocytic (granulocytic) leukemia and chronic lymphocytic leukemia), polycythemia vera, lymphoma (Hodgkin's disease and non-Hodgkin's disease), multiple myeloma, Waldenstrom's macroglobulinemia, and heavy chain disease.

Tumor cell resistance to chemotherapeutic agents represents a major problem in clinical oncology. In some embodiments, compositions and methods of the present invention provide means of ameliorating this problem by effectively administering a combined therapy approach. However, it should be noted that traditional combination therapy may be employed in combination with the compositions of the present invention. For example, in some embodiments of the present invention, immunotoxins are used before, after, or in combination with the traditional therapies.

To kill cells, inhibit cell growth, or metastasis, or angiogenesis, or otherwise reverse or reduce the malignant phenotype of tumor cells using the methods and compositions of the present invention in combination therapy, one contacts a “target” cell with the compositions described herein and at least one other agent. These compositions are provided in a combined amount effective to kill or inhibit proliferation of the cell. This process may involve contacting the cells with the immunotherapeutic agent and the agent(s) or factor(s) at the same time. This may be achieved by contacting the cell with a single composition or pharmacological formulation that includes both agents, or by contacting the cell with two distinct compositions or formulations, at the same time.

Alternatively, immunotoxin treatment precedes or follows the other agent treatment by intervals ranging from minutes to weeks. In embodiments where the other agent and immunotherapy are applied separately to the cell, one would generally ensure that a significant period of time did not expire between the time of each delivery, such that the immunotoxin and chemotherapeutic agent would still be able to exert an advantageously combined effect on the cell. In such instances, it is contemplated that cells are contacted with both modalities within about 12-24 hours of each other and, more preferably, within about 6-12 hours of each other, with a delay time of only about 12 hours being most preferred. In some situations, it may be desirable to extend the time period for treatment significantly, however, where several days (2 to 7) to several weeks (1 to 8) lapse between the respective administrations.

In some embodiments, more than one administration of the immunotherapeutic composition of the present invention or the other agent is utilized. Various combinations may be employed, where the immunotoxin is “A” and the other agent is “B”, as exemplified below:

A/B/A, B/A/B, B/B/A, A/A/B, B/A/A, A/B/B, B/B/B/A, B/B/A/B,

A/A/B/B, A/B/A/B, A/B/B/A, B/B/A/A, B/A/B/A, B/A/A/B, B/B/B/A,

A/A/A/B, B/A/A/A, A/B/A/A, A/A/B/A, A/B/B/B, B/A/B/B, B/B/A/B.

Other combinations are contemplated. Again, to achieve cell killing, both agents are delivered to a cell in a combined amount effective to kill or disable the cell.

In some embodiments of the invention, one or more compounds of the invention and an additional active agent are administered to a subject, more typically a human, in a sequence and within a time interval such that the compound can act together with the other agent to provide an enhanced benefit relative to the benefits obtained if they were administered otherwise. For example, the additional active agents can be co-administered by co-formulation, administered at the same time or administered sequentially in any order at different points in time; however, if not administered at the same time, they should be administered sufficiently close in time so as to provide the desired therapeutic or prophylactic effect. In some embodiments, the compound and the additional active agents exert their effects at times which overlap. Each additional active agent can be administered separately, in any appropriate form and by any suitable route. In other embodiments, the compound is administered before, concurrently or after administration of the additional active agents.

In various examples, the compound and the additional active agents are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other examples, the compound and the additional active agents are administered concurrently. In yet other examples, the compound and the additional active agents are administered concurrently by co-formulation.

In other examples, the compound and the additional active agents are administered at about 2 to 4 days apart, at about 4 to 6 days apart, at about 1 week part, at about 1 to 2 weeks apart, or more than 2 weeks apart.

In certain examples, the inventive compound and optionally the additional active agents are cyclically administered to a subject. Cycling therapy involves the administration of a first agent for a period of time, followed by the administration of a second agent and/or third agent for a period of time and repeating this sequential administration. Cycling therapy can provide a variety of benefits, e.g., reduce the development of resistance to one or more of the therapies, avoid or reduce the side effects of one or more of the therapies, and/or improve the efficacy of the treatment.

In other examples, one or more compound of some embodiments of the present invention and optionally the additional active agent are administered in a cycle of less than about 3 weeks, about once every two weeks, about once every 10 days or about once every week. One cycle can comprise the administration of an inventive compound and optionally the second active agent by infusion over about 90 minutes every cycle, about 1 hour every cycle, about 45 minutes every cycle, about 30 minutes every cycle or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

Courses of treatment can be administered concurrently to a subject, i.e., individual doses of the additional active agents are administered separately yet within a time interval such that the inventive compound can work together with the additional active agents. For example, one component can be administered once per week in combination with the other components that can be administered once every two weeks or once every three weeks. In other words, the dosing regimens are carried out concurrently even if the therapeutics are not administered simultaneously or during the same day.

The additional active agents can act additively or, more typically, synergistically with the inventive compound(s). In one example, one or more inventive compound is administered concurrently with one or more second active agents in the same pharmaceutical composition. In another example, one or more inventive compound is administered concurrently with one or more second active agents in separate pharmaceutical compositions. In still another example, one or more inventive compound is administered prior to or subsequent to administration of a second active agent. The invention contemplates administration of an inventive compound and a second active agent by the same or different routes of administration, e.g., oral and parenteral. In certain embodiments, when the inventive compound is administered concurrently with a second active agent that potentially produces adverse side effects including, but not limited to, toxicity, the second active agent can advantageously be administered at a dose that falls below the threshold that the adverse side effect is elicited.

Other factors that may be used in combination therapy include, but are not limited to, factors that cause DNA damage such as γ-rays, X-rays, and/or the directed delivery of radioisotopes to tumor cells. Other forms of DNA damaging factors are also contemplated such as microwaves and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments of the present invention, the regional delivery immunotoxins to patients with cancers is utilized to maximize the therapeutic effectiveness of the delivered agent. Similarly, the chemo- or radiotherapy may be directed to a particular, affected region of the subject's body. Alternatively, systemic delivery of the immunotherapeutic composition and/or the agent may be appropriate in certain circumstances, for example, where extensive metastasis has occurred.

In addition to combining the immunotoxins of some embodiments of the present invention with chemo- and radiotherapies, it also is contemplated that traditional gene therapies are used. For example, targeting of p53 or p16 mutations along with treatment with the immunotoxins of the present invention provides an improved anti-cancer treatment. The present invention contemplates the co-treatment with other tumor-related genes including, but not limited to, p21, Rb, APC, DCC, NF-I, NF-2, BCRA2, p16, FHIT, WT-I, MEN-I, MEN-II, BRCA1, VHL, FCC, MCC, ras, myc, neu, raf erb, src, fms, jun, trk, ret, gsp, hst, bcl, and abl.

An attractive feature of the present invention is that the therapeutic compositions may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (SCIMED LIFE SYSTEMS, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

In some embodiments, composition embodiments of the present invention are co-administered with an anti-cancer agent (e.g., chemotherapeutic). In some embodiments, method embodiments of the present invention encompass co-administration of an anti-cancer agent (e.g., chemotherapeutic). The present invention is not limited by type of anti-cancer agent co-administered. Indeed, a variety of anti-cancer agents are contemplated to be useful in the present invention including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda).

Other anti-cancer agents include: Antiproliferative agents (e.g., Piritrexim Isothionate), Antiprostatic hypertrophy agent (e.g., Sitogluside), Benign prostatic hypertrophy therapy agents (e.g., Tamsulosin Hydrochloride), Prostate growth inhibitor agents (e.g., Pentomone), and Radioactive agents: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

Another category of anti-cancer agents is anti-cancer Supplementary Potentiating Agents, including: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca++ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

Still other anticancer agents are those selected from the group consisting of: annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO4; mitoxantrone; mitozolomide; Pentostatin; and Tomudex.

Other cancer therapies include hormonal manipulation. In some embodiments, the anti-cancer agent is tamoxifen or the aromatase inhibitor arimidex (i.e., anastrozole).

In some embodiments, the additional agent is Mitomycin C.

### EXPERIMENTAL

The following examples are provided in order to demonstrate and further illustrate certain preferred embodiments and aspects of the present invention and are not to be construed as limiting the scope thereof.

### Example 1

### A. Materials and Methods

Clinical Samples

The clinical samples used for establishment of the animal models from human tumor tissues was previously described (Flatmark et al, Hum Pathol 2010; 41:1109-19; Flatmark et al., BMC Cancer 2007; 7:116.). In the present work, mucinous tumor tissue harvested at the time of surgery from two additional patients with clinical pseudomyxoma peritonei, was used in short-term cultures to study ex vivo immunotoxin efficacy and cell death mechanisms. The study was approved by the regional ethics board of south-east Norway and written informed consent was obtained from the patients. The PMP-3 sample was obtained from a 40-year old woman who previously had been subjected to appendectomy (for a ruptured mucinous cystadenoma) and bilateral salpingo-oophorectomy (for ovarian recurrence), 7 and 2 years previously. A wide-spread intraabdominal recurrence was detected, histologically classified as peritoneal mucinous carcinomatosis of intermediate features (according to the Ronnett classification (Cancer 2001; 92:85-91)). The PMP-6 sample, also harvested at the time of cytoreductive surgery, was from a 41-year old male who one year previously had an appendectomy for a mucinous cystadenoma of the appendix which ruptured during surgery. He presented with an extensive peritoneal recurrence, histologically classified as disseminated peritoneal adenomucinosis. Both patients were subjected to extensive cytoreductive surgery, accomplishing complete cytoreduction, and they received hyperthermic i.p. chemotherapy with MMC.

Drugs

Stock solutions of MMC (Medac, Wedel, Germany) and OXA (Sanofi Aventis, Paris, France) were prepared in 5% glucose (Fresenius Kabi AB, Uppsala, Sweden) and stored as recommended by the drug manufacturers. The MOC31 antibody (MCA Development, Groningen, the Netherlands) detects the epithelial glycoprotein EpCAM. The immunotoxin was constructed by conjugating the MOC31 antibody to pseudomonas exotoxin, (PE) (obtained from Dr Darrel Galloway (University of Ohio, Columbus, Ohio) by a thioether bond formed with the reagent sulfo-SMCC (sulfo-succinimidyl-4-(N-maleimidomethyl)cyclohexane-1-carboxylate) (Pierce, Rockford, Ill.) as described earlier (Godal et al., Int J Cancer 1992; 52:631-5). Clinical grade MOC31PE was produced at Fred Hutchinson Cancer Centre (Seattle, Calif.) and was dissolved and diluted (when applicable) in PBS with 0.1% human serum albumin. For animal experiments, stock solutions of MMC and OXA were diluted in 5% glucose (Fresenius Kabi AB, Uppsala, Sweden).

Animal Experiments

The establishment and propagation of the five human orthotopic models of pseudomyxoma peritonei and mucinous peritoneal carcinomatosis was previously described (Flatmark et al., 2007, supra; Flatmark et al., 2010, supra). Briefly, the tumor models were established by implanting human mucinous tumor tissue from patients undergoing cytoreductive surgery of the peritoneal cavity. The macroscopic growth pattern of the models closely resembled their human counterparts, with strict i.p. growth without metastasis development. The tumor tissue, microscopically dominated by large amounts of extracellular mucin with clusters of tumor epithelium, could be harvested as liquiform, highly viscous mucinous tumor tissue and injected i.p. for passage to new generations of mice or used for ex vivo experiments. The presence of tumor tissue was asymptomatic in the mice, to be detected only by visible abdominal distension, which was used as humane end point. In-house bred BALB/c (nu/nu) female mice, age 4-6 weeks were used. Animals were kept under specific pathogen-free conditions, and food and water were supplied ad libitum. Housing and all procedures involving animals were performed according to protocols approved by the animal care and use committee.

For the ex vivo studies, mucinous tumor tissue was harvested at autopsy and immediately brought to the laboratory for further processing as described in the respective sections. For the in vivo studies, 200-250 μl mucinous ascites was injected i.p. in each study animal. Animals were randomly assigned to treatment groups (in most cases 6 animals per group), and the next day, or on day 6, the study drugs (MMC, OXA or MOC31PE) or vehicle (5% glucose) were administered in the form of a single i.p. injection. Additional control experiments were performed by i.p. administration of MOC31 alone (without the toxin moiety), and another isotype-like (IgG1) antibody (BM7; performed in the PMCA-3 model) as well as human gamma globulin, which contains a mixture of immunoglobulin species (#G 4386, Sigma Aldrich, Saint Louis, Mo., USA; PMCA-2, PMP-2 and PMCA-3 models), and no growth inhibition was observed compared to vehicle treatment. The BM7 antibody, which recognizes a glycosylated epitope on the MUC1, was provided by Dr S Kaul (Frauenklinik, Heidelberg, Germany). Animals were monitored for symptoms of drug toxicity and for the presence of tumor growth. As tumor growth does not give rise to symptoms of disease in these models, animals were sacrificed by cervical dislocation when and if i.p. tumor growth was detected in the form of abdominal distention, or at the end of the experiment (which was defined to be at least 2× the observation period of untreated animals). At autopsy, tumor was collected and weighed, and if possible, tumor samples from at least two animals in each treatment group were collected. Samples were formalin fixed, paraffin-embedded, sectioned, and haematoxylin-eosin stained, and the presence of typical tumor tissue for each model was verified. Very few animals had to be sacrificed for other reasons than tumor growth and they were excluded from analysis.

Calculation of Growth Index

Because tumor growth was asymptomatic and was evaluated by visual inspection, the observation time and amount of harvested tumor at autopsy individually were insufficiently accurate as outcome parameters, and an alternate approach was worked out to express tumor growth in each animal. A growth index was devised to calculate an end-point for each animal, combining these two key parameters, final tumor load (tumor weight) and the time for each tumor to reach its final volume relative to the total duration of the experiment:

Growth index=Tumor weight+(T_{Total}−T_{A})/T_{Total}×10 in which T_{Total} is the entire duration of the experiment in days; T_{A} is the time from start of the experiment until sacrifice of the specific animal. The fraction was multiplied by 10 to balance the relative contribution of the time factor with that of tumor load at autopsy.

Example of how the growth index is calculated using the equation:

Mouse 1 (Vehicle): Killed on day 50 (T_{A}), and 4 g of tumor tissue was harvested

Mouse 2 (Very efficacious drug): Killed on day 100 (T_{A}), 0.3 g of tumor tissue harvested

Mouse 3 (Not so efficacious drug): Killed on day 90 (T_{A}), 4 g of tumor tissue harvested

The experiment lasted for 100 days, which gives the T_{Total} (defined as at least 2 times the observation period of the vehicle group). Respective growth indices would then be:

Mouse 1: 4+((100−50)/100)*10=4+5=9

Mouse 2: 0.3+((100−100)/100)*10=0.3+0=0.3

Mouse 3: 4+((100−90)/100)*10=4+1=5

Immunohistochemistry

Frozen tissue sections were air dried and stored at −80° C. Buffers, streptavidin-horseradish peroxidase (HRP) and 3′,3′-diaminobenzidine (DAB) substrate used for immunochemistry were all from DAKO (Glostrup, Denmark). After thawing, the slides were fixed for 10 min in ice-cold acetone, washed with buffer and blocked for 10 min at room temperature (RT) with biotin blocking system part one. After washing, the sections were incubated for 10 min at RT with biotin blocking system part two. After washing, sections were incubated with biotinylated human EpCAM antibody (kind gift from Affitech AS, Oslo, Norway) (16 μg/ml in ChemMate antibody diluents) overnight at 4° C. The human anti-EpCAM antibody was biotinylated as recently described.¹⁵ Next day the sections were washed, incubated with streptavidin-HRP complex, for 1 h at RT and subsequently washed before the sections were stained with DAB+chromatin substrate, counterstained with hematoxylin and mounted. Negative control sections were treated as described above except for the incubation with primary antibody.

Cell Viability Assay

For the ex vivo experiments, (cell viability, protein synthesis and Western Blot analysis), mucinous tumor tissue from animals or patients was diluted 1:4 and passaged through decreasing dimensions of syringes until a homogenous cell suspension was obtained. Because of the high mucin content, the cell suspension was highly viscous, and determining the number of cells seeded was not possible, and standardization was achieved by using the same volume of cell suspension through each experimental series. Using the PMCA-2 model as an example, analyzing ex vivo cell viability of vehicle treated cells, mean absorbance was 0.986+/−0.067, representing a SD of 6.8% (in 3 separate biological experiments with 3-6 parallels), and in vivo, the variability was on a similar scale (SD 7.8%). The observed differences between treatment groups were thus deemed to clearly transcend intra-experimental variability indicating that using tumor volume as a means of assay standardization was acceptable. For the cell viability assay, the suspension was seeded in 96-well plates (100 μl/well), MOC31PE (1000 ng/ml) or vehicle was added and the cells were incubated in a standard tissue culture incubator at 37° C. for 24 h. Solution Cell Proliferation Assay (Promega, Madison, Wis.) was then added to the wells to quantify the presence of viable cells, and absorbance was measured 1-2 h later at a wavelength of 490 nm in a Wallac Victor 2 plate reader (Perkin Elmer, Waltham, Mass.). After correcting for background absorbance, values generated for treated cells and untreated controls were compared and percentage cell viability was calculated. Experiments were set up with 3-6 parallel samples, and experiments with xenograft tissue were repeated at least three times, whereas analyses of live cells from patient samples could be performed only once.

Protein Synthesis Assay

Protein synthesis was measured by using the [³H]-leucine incorporation assay (Sandvig 1982). Mucinous cell suspension was seeded in 48-well plates (250 μl/well) and MOC31PE (1-1000 ng/ml) or vehicle was added immediately and the suspension was incubated at 37° C. for 24 h. Afterwards, cells were washed twice with cold PBS with 0.1% human serum albumin, and incubated with [³H]-leucine (2 μCi/ml⁻¹) in leucine-free medium for 60 min at 37° C. The cells were then washed twice with 5% trichloroacetic acid for 10 and 5 min, respectively, and dissolved in 0.1 M KOH. The resultant solution was transferred to the liquid scintillator Aquasafe 300 Plus (Zinsser Analytic, Frankfurt/Main, Germany) Sample counts were determined in a liquid scintillation counter (LKB Wallac, Perkin Elmer, Boston, Mass., USA). Assays were performed in triplicate, and repeated three times for PMP-2 and once for PMCA-2.

Western Blot Analysis

The mucinous cell suspension was seeded in 48-well plates (500 μl/well). MOC31PE (1000 ng/ml) or vehicle was immediately added and the cells were incubated at 37° C. for 24 h. Thereafter, the cells were lysed by using an SDS boiling method as described previously (Andersson2009). In brief, cell pellets were resuspended in lysis buffer (2% SDS, 1 mM Na₃VO₄, and 10 mM Tris-Cl (pH 7.6)), which was held at 100° C. when added, and the lysates were boiled for 5 min. After six passages through a 20G syringe on ice, the lysates were cleared by centrifugation. Protein concentrations were then determined using the BCA (bicinchoninic acid) protein assay (Pierce, Rockford, Ill.). The lysates were snap frozen in liquid N₂ and kept at −70° C. A portion (15 μg) of each protein lysate was fractionated by 4-12% NuPAGE Bis-Tris gel electrophoresis (Invitrogen, Carlsbad, Calif.), and subsequently transferred by electroblotting to Immobilon membranes (Millipore, Bedford, Mass.). The filters were probed with an anti-PARP antibody (Roche Diagnostics, Mannheim, Germany), anti-EpCAM (MOC31, IQ Corporation, Groningen, the Netherlands) or with anti-alpha tubulin (Calbiochem, La Jolla, Calif.). Immune complexes were detected with an appropriate HRP-coupled secondary antibody. Peroxidase activity was visualized with enzyme-linked chemiluminescence (Amersham Pharmacia Biotech, Buckinghamshire, UK). The western blots were probed with anti-α-tubulin antibody to confirm equal loading and transfer of samples.

Statistical Analyses

Student t-tests were performed to compare treatment groups in the various assays, using SPSS (Statistical Package for Social Sciences) version 16.0 (SPSS Inc., Chicago, Ill.), and p-values below 0.05 were considered statistically significant.

### B. Results

In Vivo Tumor Growth Inhibition by MMC and OXA

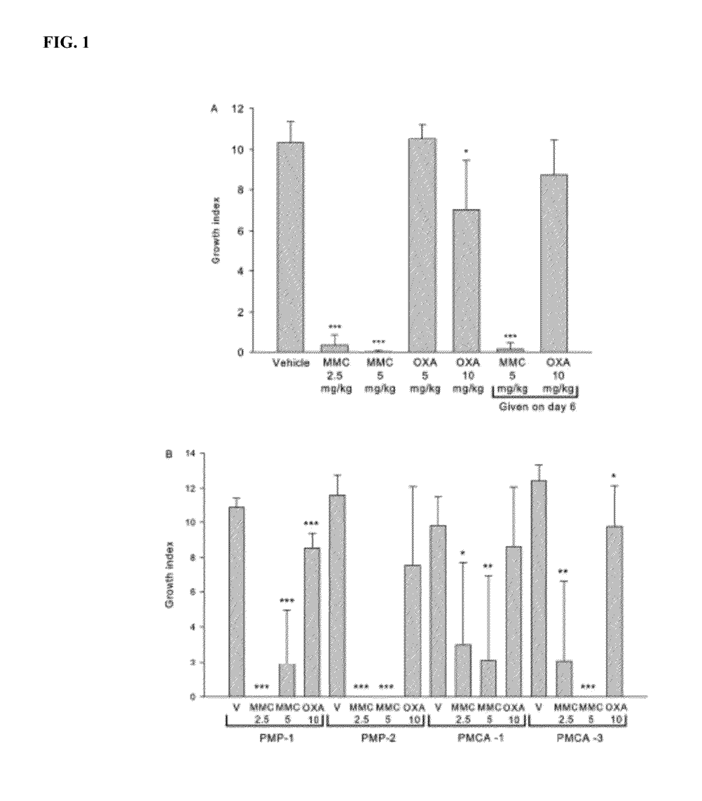

Studies of tumor growth inhibition by MMC and OXA were undertaken to examine how the model systems would perform, comparing two drugs with expected efficacy since they both are in clinical use for treatment of mucinous peritoneal surface malignancies. Tumor take was in all models 100% in control animals. As shown in Table 1, the mean time period for control animals to develop visible abdominal distension ranged between 38 days for the PMP-2 model and 46 days for the PMCA-1 and PMP-1 models. The mean amount of mucinous tumor tissue harvested per tumor-bearing mouse in untreated animals ranged between 4.0 and 6.4 g, the variability reflecting difficulty of reproducibly evaluating the degree of abdominal distention in otherwise asymptomatic animals. Several series of experiments were performed to establish relevant dose levels and toxicities using the PMCA-2 model. For MMC, doses of 5 mg/kg were well tolerated, whereas toxicity (failure to thrive or weight loss in the absence of tumor development) was observed at 10 mg/kg, while the highest tolerated dose of OXA was 10 mg/kg (data not shown). Within the tolerated dose levels of MMC, 5 mg/kg almost completely inhibited tumor growth, as only one of 11 animals developed tumor during the observation period, and only a minimal amount of tumor was collected, giving a mean growth index of 0.02. A single dose of 2.5 mg/kg was slightly less efficacious, giving a mean growth index of 0.3 (FIG. 1). In contrast, the highest tolerable dose of OXA (10 mg/kg) only slightly delayed tumor growth in the PMCA-2 model, giving a mean growth index of 7.0, and at 5 mg/kg the mean growth index was equal to that of untreated animals. Experiments were also performed to assess whether the effects were confined to the immediate period after tumor implantation, and delaying treatment start until day 6, using the highest tolerable doses for each drug, gave very similar results compared to treatment given on day 1.

Based on these results, in the remaining four models 2.5 mg/kg and 5 mg/kg of MMC and 10 mg/kg OXA were used. In spite of variability between the models, some trends were observed. In all models, growth inhibition induced by OXA was clearly inferior to that of MMC, as OXA gave rise to only slight reduction of growth indices compared to vehicle-treated animals (FIG. 1). In the PMP-1 and PMP-2 models, both tested doses of MMC almost completely inhibited tumor growth within the observation period, and for PMCA-1 and PMCA-3, 5 mg/kg was more efficacious than 2.5 mg/kg MMC.

Expression of EpCAM in Xenografts and Patient Tissues

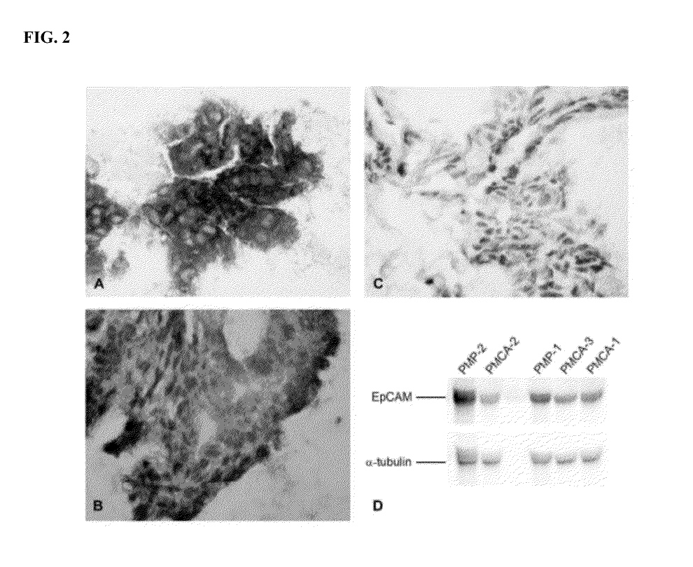

A prerequisite for optimal immunotoxin activity is the presence of the target antigen on the surface of target cells. Expression of EpCAM was detected by immunohistochemistry in tumor cells in samples from all the animal models. FIG. 2 A-C depicts the results for the PMCA-2 and PMP-2 models, indicating the presence of EpCAM in the cytoplasm and on the cell membrane of tumor cells. Additionally, western immunoblot analysis was performed on tissue lysates from xenografts using the mouse monoclonal MOC31 antibody, confirming presence of EpCAM in all examined samples (FIG. 2D).

In Vivo Tumor Growth Inhibition by MOC31PE and MMC

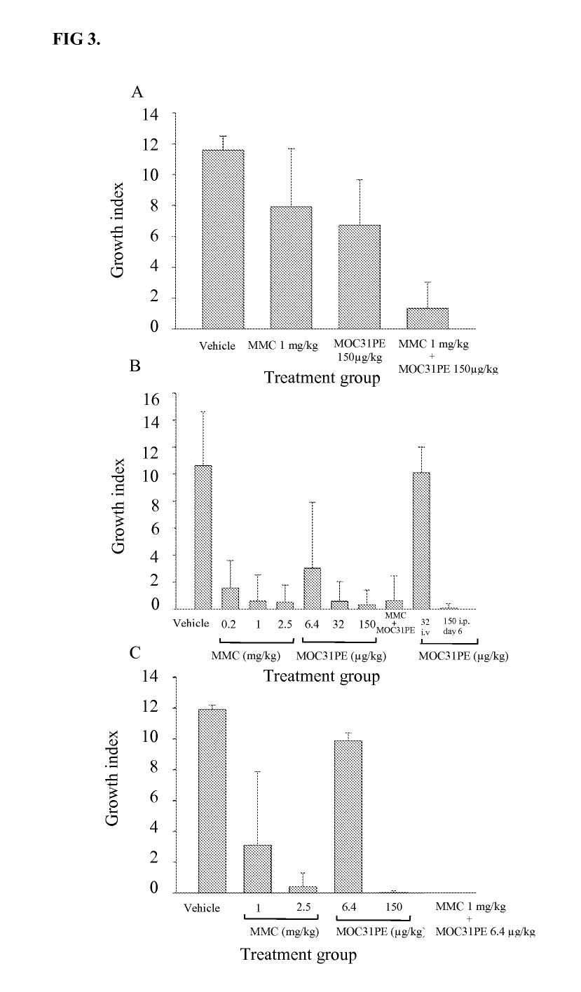

In a new set of animal experiments, the efficacy of administering single i.p. injections of MOC31PE and MMC alone or in combination was assessed in two of the animal models. In the PMCA-2 model, MMC 1 mg/kg inhibited tumor growth, but clearly less effectively than the higher doses (2.5 and 5 mg/kg) used in the model validation series. MOC31PE (150 μg/kg, previously determined as maximum tolerable i.p. dose in this strain of mice, data not shown) similarly brought about a clear reduction in growth index, while combination of the two drugs in the same doses had at least additive effect, almost completely preventing tumor growth in the animals (FIG. 3A). The PMP-2 model was more sensitive to both MMC and MOC31PE, as all tested doses resulted in substantial inhibition of tumor growth in this model (FIG. 3B), and even the lowest immunotoxin dose (6.4 μg/kg) resulted in more than 50% growth inhibition. Similarly to the validation studies, delaying treatment start until day 6 gave almost equal results compared to day 1 administration. No advantage of combining the drugs could be detected for the tested drug combinations (MMC 1 mg/kg+MOC31PE 150 μg/kg; MMC 1 mg/kg+MOC31PE 6.4 μg/kg; MMC 0.2 mg/kg+MOC31PE 6.4 μg/kg). Intravenous injection of a low dose of MOC31PE (32 μg/kg) did not have any effect on i.p. tumor growth, indicating that the effect was caused by a local interaction in the peritoneal cavity. The PMCA-3 model exhibited intermediate sensitivity towards both drugs (FIG. 3C). Combining the two lowest drug doses given (MMC 1 mg/kg+MOC31PE 6.4 μg/kg) completely prevented tumor growth in the PMCA-3 model.

Ex Vivo Activity of MOC31PE

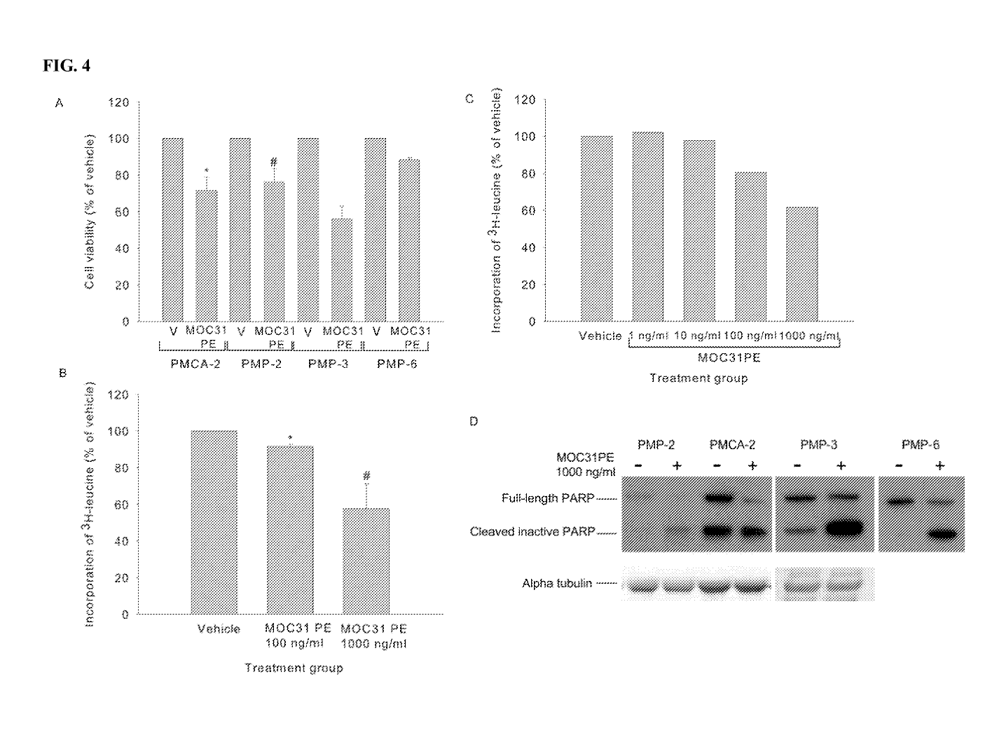

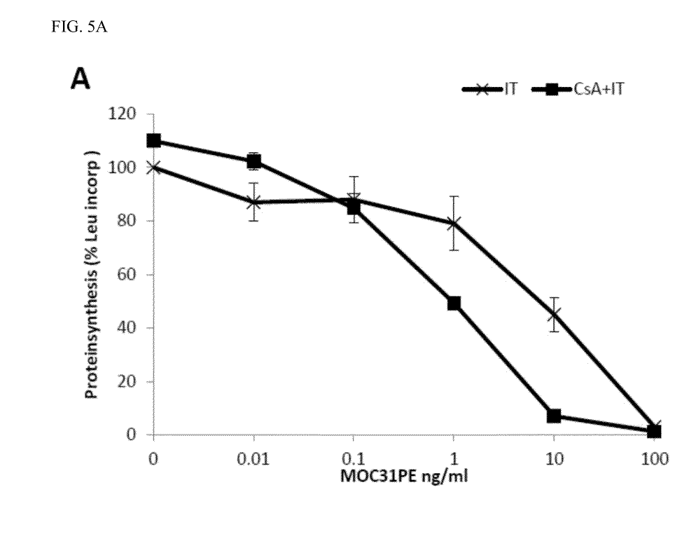

To investigate immunotoxin activity, short-term (24 h) ex vivo experiments were performed using tumor tissue harvested either from tumor-bearing animals (PMCA-2 and PMP-2) or taken directly from the operating theatre from patients undergoing surgery (PMP-3 and PMP-6). Ex vivo treatment of mucinous tumor tissue with MOC31PE (1000 ng/ml) for 24 h resulted in approximately 25% reduction of cell viability in both tumor models compared to vehicle-treated cells (FIG. 4A). Ex vivo activity of the immunotoxin was also observed in the patient samples, with 44% and 12% reduction of cell viability for the PMP-3 and PMP-6 models, respectively.

Mechanisms of Immunotoxin Activity

Inhibition of protein synthesis has been shown to be one of the main mechanisms involved in immunotoxin-related cell death, and in the ex vivo setting, a dose response relationship was present when mucinous tumor tissue was treated with MOC31PE in both models (FIGS. 4 B and 4C). Protein synthesis was decreased by approximately 20% and 40% after 24 h treatment with MOC31PE 100 ng/ml or 1000 ng/ml, respectively.

Another typical feature of immunotoxin mediated cell death is the induction of apoptosis, which can be visualized by the detection of poly (ADP-ribose) polymerase (PARP) cleavage by Western immunoblot analysis. Upon ex vivo treatment of mucinous tumor tissue with MOC31-PE immunotoxin, PARP cleavage was detected in PMP-2 xenograft tissue as well as in both samples taken directly from patients. In the PMCA-2 model the 85 kDa cleavage product was present in the untreated sample at a relatively substantial level, and no increase was detected after immunotoxin treatment, although depletion of the uncleaved form was detected, indicating that apoptosis-related cleavage still had taken place (FIG. 4 D). No PARP inactivation was evident in cells treated with the MOC31 antibody alone or with the PE toxin alone, showing that the effect was solely dependent on the specific cytotoxic effect of MOC31PE.

The rationale for applying local therapy (cytoreductive surgery and i.p. chemotherapy) in peritoneal surface malignancies rests on the conception of the peritoneal cavity as a unique compartment, with the peritoneum as a barrier against local invasion and metastatic tumor spread.¹⁶ Thus, even if cancerous dissemination has occurred widely in the peritoneal cavity, complete tumor eradication can accomplished provided the peritoneal barrier has not been compromised. The possibility of adding immunotoxin treatment to standard therapy is particularly appealing, since it provides direct cytotoxicity and has a unique mechanism of action compared to other cytotoxic drugs.

Differential sensitivity towards MMC and OXA was observed in all five models, in all cases favoring MMC over OXA, using the highest doses tolerated by the mice. Only one patient (from whom tissue was used to establish the PMCA-2 model) had previously received chemotherapy, in the form of adjuvant 5-fluorouracil after the primary procedure and neoadjuvant 5-fluoruracil in combination with OXA upon diagnosis of peritoneal carcinomatosis. MMC and OXA are the drugs most extensively used in i.p. chemotherapy for mucinous peritoneal surface malignancies, MMC being the preferred drug in pseudomyxoma peritonei, whereas OXA is being studied as an alternative to MMC in the treatment of peritoneal carcinomatosis from colorectal cancer (Elias et al., J Clin Oncol 2010; 28:63-8). In the human treatment setting, i.p. therapy is directed towards eradication of low-volume disease, and particularly against exfoliated tumor cells in the peritoneal cavity. Delivering treatment on day 1 in the models was aimed to mimic this situation, but the same differences were observed when the drugs were administered on day 6 (Table 1), indicating that the effects were not confined to the immediate period after tumor implantation. Thus, the results indicate that the current orthotopic models of mucinous peritoneal surface malignancies can be effectively used to study treatment efficacy of i.p. administered drugs.

Although all three tested models were sensitive to MMC and MOC31PE, they exhibited very different sensitivity towards each drug given alone and in combination. In the PMCA-2 model, moderate effects were observed with MMC and MOC31PE alone, while the combination was significantly more efficacious than either drug given as monotherapy. In contrast, in the PMP-2 model, all tested drug doses were efficacious, and no additional benefit was gained from combining the drugs, indicating that further dose reduction might be appropriate in this very sensitive model. The PMCA-3 model exhibited intermediate sensitivity towards both drugs. For MOC31PE, the best indicative factor is cell surface antigen expression, which was similar in all the models as assessed by immunohistochemistry. Thus, in addition to demonstrating the efficacy of administering MOC31PE in this setting, our results demonstrate the presence of differential drug sensitivity in peritoneal surface malignancies that currently has not been explored in human disease.

Ex vivo, MOC31PE inhibited cell viability in the PMCA-2 and PMP-2 models, and similar findings were made in the samples of mucinous tumor tissue cultured directly from two patients (PMP-3 and PMP-6). The ex vivo effects of MOC31PE were relatively modest compared with the in vivo efficacy, probably reflecting differences in microenvironment between the ex vivo culturing conditions and the peritoneal cavity in nude mice. However, the consistent effects on cell viability indicated that the ex vivo situation is appropriate for studying mechanisms for MOC31PE-mediated cell death. Both the ex vivo and the in vivo conditions aimed to simulate the therapeutic challenge encountered after cytoreductive surgery when free tumor cells without vasculature are left in the peritoneal cavity. In subsequent experiments, mechanistic evidence of immunotoxin activity was identified in the examined samples; specifically, inhibition of protein synthesis as assessed by ³H-leucine incorporation (in PMCA-2 and PMP-2) and induction of apoptosis by demonstrating PARP cleavage (additionally in PMP-3 and PMP-6) (FIG. 4).

I.p. administration after cytoreductive surgery for mucinous peritoneal malignancies of intestinal origin is an ideal setting treatment with MOC31PE. The tumor burden after cytoreductive surgery is very low and restricted to the peritoneal cavity and its surfaces, which allows optimal interaction between MOC31PE and remaining tumor cells. EpCAM was highly expressed in all the models, and the observed ex vivo effects of MOC31PE in two samples taken directly from patients shows a similarly sensitive phenotype. Most colorectal tumors express EpCAM, and MOC31PE is thus an ideal cytotoxic molecule for targeting this disease (Went et al., Br J Cancer 2006; 94:128-35). Clinical implementation of immunotoxins for cancer treatment has been hampered by neutralizing anti-immunotoxin antibody response, making repeated administrations a challenge, and in some cases by liver toxicity, neurotoxicity and vascular leak syndrome. No antagonistic interaction was observed with MMC in the present work, indicating that MOC31PE can be added to the standard treatment setting.

TABLE 1
In vivo tumor growth in five tumor models upon treatment with vehicle, mitomycin C and oxaliplatin.

| | | | Mitomycin C | | | Oxaliplatin | | |
|---|---|---|---|---|---|---|---|---|
| | | | | | Day 6 | | | Day 6 |
| | | | 2.5 | 5.0 | 5 | 5 | 10 | 10 |
| | | Vehicle | mg/kg | mg/kg | mg/kg | mg/kg | mg/kg | mg/kg |
| PMCA-2 | Tumor growth/total number | 14/14 | 1/3 | 1/11 | 1/5 | 6/6 | 10/10 | 4/4 |
| | of animals | | | | | | | |
| | Days until sacrifice (mean) | 42 | 106 | 103 | 106 | 44 | 76 | 74 |
| | Mucin (g), mean (SD) | 4.4 (1.3) | 1 (0.6) | 0.02 (0.1) | 0.1 (0.3) | 4.9 (0.8) | 4.5 (1.4) | 5.8 (1.6) |
| PMP-1 | Tumor growth/total number | 6/6 | 0/6 | 1/6 | | | 6/6 | |
| | of animals | | | | | | | |
| | Days until sacrifice (mean) | 46 | 110 | 100 | | | 68 | |
| | Mucin (g), mean (SD) | 5.0 (0.9) | 0 | 1.0 (2.3) | | | 4.7 (0.7) | |
| PMP-2 | Tumor growth/total number | 6/6 | 0/6 | 0/6 | | | 5/6 | |
| | of animals | | | | | | | |
| | Days until sacrifice (mean) | 38 | 111 | 111 | | | 74 | |
| | Mucin (g), mean (SD) | 5.1 (0.9) | 0.7 (0.4) | 0.5 (0.7) | | | 4.3 (2.3) | |
| PMCA-1 | Tumor growth/total number | 6/6 | 3/6 | 1/6 | | | 6/6 | |
| | of animals | | | | | | | |
| | Days until sacrifice (mean) | 46 | 94 | 98 | | | 68 | |
| | Mucin (g), mean (SD) | 4.0 (1.3) | 1.5 (2.3) | 1 (2.6) | | | 4.7 (2.4) | |
| PMCA-3 | Tumor growth/total number | 6/6 | 4/6 | 0/5 | | | 6/6 | |
| | of animals | | | | | | | |
| | Days until sacrifice (mean) | 45 | 102 | 112 | | | 63 | |
| | Mucin (g), mean (SD) | 6.4 (0.7) | 1.2 (2.4) | 0 | | | 5.3 (1.2) | |

### Example 2

### Materials and Methods

Materials

RPMI-1640, PBS, Glutamax, and Hepes were obtained from Lonza (Austria). Fetal calf serum was purchased from PAA (GE Healthcare, UK), MEM w/o leucine, 0.25% Trypsin/EDTA from Gibco, and YoYo-1 fluorescent dsDNA staining from Molecular Probes (Life Technologies, UK), and tritiated Leucine from Perkin Elmer (Waltham, Mass.). Cyclosporine A was purchased from Calbiochem (San Diego, Calif.) and dissolved in ethanol to 8.3 mM stock solution. The GenElute Mammalian total RNA kit and general laboratory chemicals were from Sigma Aldrich (St. Louis, Mo.), the Cell Titer 96 AqueousOne solution (MTS) cell proliferation assay was from Promega (Madison, Wis.). RT² Profiler PCR Array System, including the cDNA synthesis kit, and SYBR green were from SABiosciences (Qiagen Nordic). Chemicals for validation of gene expression were from Applied (Life Technologies, UK). Plastic ware for cell culture was from Nunc (Thermo Scientific), gels and buffers for protein electrophoresis from Life Technologies, HRP-conjugated antibodies from Dako (DK), and chemiluminescent super-signal substrate from Thermo Scientific.

Cells and Immunotoxin

The human ovarian cancer cell lines B76 (Marth et al., Int J Cancer 1996, 67: 826-830) and HOC-7 (Buick et al., Cancer Research 1985, 45: 3668-3676) were a gift from Dr C. Marth (Innsbruck Medical University, Innsbruck, Austria). In this study B76 was our main cell line and HOC-7 was used to confirm key results. The cell lines were cultivated in RPMI 1640 medium added Glutamax, Hepes and 8% heat-inactivated fetal calf serum. The monoclonal antibody MOC31 (Myklebust et al., Br J Cancer 1991, 63: 49-53) binds epithelial cell adhesion molecule (EpCAM, CD326) and was conjugated to whole Pseudomonas exotoxin A as previously described (Engebraaten et al., Int J Cancer 2000, 88: 970-976).

Protein Synthesis and Cell Viability

The [³H]-leucine incorporation assay was used to quantify protein synthesis (Engebraaten et al., Int J Cancer 2000, 88: 970-976) and the Cell Titer 96 AqueousOne solution (MTS) assay was used to determine cell viability as previously described (Andersson et al., Molecular Cancer Research 2006, 5: 1050-1059).

Cell Proliferation, Membrane Damage and Scratch-Wound Healing in the IncuCyte

Cells were seeded in 96 well plates and grown to ≈50% confluency, transferred to the IncuCyte (Essen BioSciences, Ann Arbor, Mi) after the medium was replaced with fresh medium with or without IT and/or CsA. Membrane damage was measured after adding YoYo-1, a dye that emit fluorescence when it binds to double-stranded DNA. The cytotoxic index is defined as the number of fluorescent objects in a well, divided by the total number of fluorescent objects obtained after 0.1% Triton X-100 is added to open all cells in the well. For migration studies, the wound maker tool was used to make scratch wounds in confluent cell culture monolayers in 96 well image-lock plates (Essen BioSciences). Plates were incubated in the IncuCyte for 24 h and an integrated metric called relative wound density (RWD) was used to quantify effects on migration. This metric measures the cell density in the wound area relative to the cell density outside the wound area.

RNA Isolation and PCR Array Analyses

The cells were seeded in 6 well plates, grown to ≈80% confluency and treated for 24 h before RNA was isolated from adherent cells using the GenElute Mammalian total RNA kit (Sigma Aldrich) and quantified in a Picodrop spectrophotometer (Picodrop Ltd, UK). RNA isolated for PCR array assays was treated with DNase I (Invitrogen) and the RNA quality was checked in the UV spectrophotometer. For cDNA synthesis (1 μg/reaction) the RT² first strand kit from SABiosciences was used. The resulting cDNA was diluted and qPCR was run as described in the PCR array protocol (SABiosciences RT² Profiler PCR Array System) using a BioRad ICycler. Gene expression was tested using either Cancer Pathway Finder (untreated, IT 10 ng/ml)—or Tumor Metastasis (2 μM CsA, CsA+IT 10 ng/ml) array. There are primers for 84 test genes and 5 reference genes (B2M, HPRT1, RPL13A, GAPDH, and ACTB) on each 96-well plate. Data analysis was performed as described in the protocol from the manufacturer and by using their PCR Array Data Analysis Web portal.

Validation of PCR Array Data

Gene expression was validated in independent experiments with RNA isolated as described above. The high capacity RNA to DNA master mix was used for cDNA synthesis (1 μg RNA/reaction). Gene expression was measured using qPCR analyses with TaqMan probes using the 7500 Real Time PCR machine (Applied Biosystems). Each sample was tested in duplicate. Fold change in expression was calculated using the comparative Cₜ method with RPL37A as a reference gene since the expression of this gene was similar in control and experimental groups. The gene list and corresponding probes are shown in Additional table 1.

Subcellular Fractionation, Gel Electrophoresis, and Antigen Detection.

Cells were grown to 70-80% confluency in 75 cm² flasks and treated with MOC31PE and/or CsA for 24 h. The cells were washed with cold PBS and lysed in 500 μl SF buffer (250 mM sucrose, 20 mM Hepes, 10 mM KCl, 1.5 mM MgCl₂, 1 mM EDTA and 1 mM EGTA, pH 7.5) and the protease inhibitor cocktail was added (MiniComplete, Roche). Cells were scraped from the flasks and the lysates were passed through 25G needles 10 times, and incubated on ice for 20 min. The nuclear pellet was centrifuged out at 720 g for 5 min and the resulting supernatant centrifuged at 10000 g for 10 min to separate the cytosolic (supernatant) and mitochondrial (pellet) fractions. Pellets were washed with 500 μl SF buffer, passed through 25G needles 10 times and re-centrifuged. Finally, the pellets were resuspended in 50 μl lysis buffer (10 mM Tris pH 7.5, 1% SDS, 1 mM Na₃VO₄, 0.1% Triton X-100, and 10% glycerol) and briefly sonicated. For total cell lysates, cells were lysed in boiling lysis buffer as previously described (Andersson et al., 2006, supra). Proteins were resolved on 4-12% Nu-PAGE gels and blotted onto PVDF membranes for antigen detection. The purity of the fractions was validated with antibodies detecting atubulin (cytosol, Cell Signaling), lamin B1 (nucleus, Abcam), and F₁F_{O}-ATP synthase (mitochondria, Calbiochem). NR4A3 in the fractions was detected on separate blots using a polyclonal anti-NR4A3/NOR-1 antibody (Novus Biologicals). Chemiluminescence signals were recorded using the G:Box system with a CCD camera from SynGene and quantified using the provided GeneTool software.

Statistical Analyses

Statistical significance was evaluated with two-tailed Students t-test except for qPCR validations where non-parametric Mann-Whitney tests were used. In both tests p-values at 0.05 were considered statistically significant.

Results

MOC31PE Immunotoxin Inhibits Protein Synthesis and Reduces Cell Viability

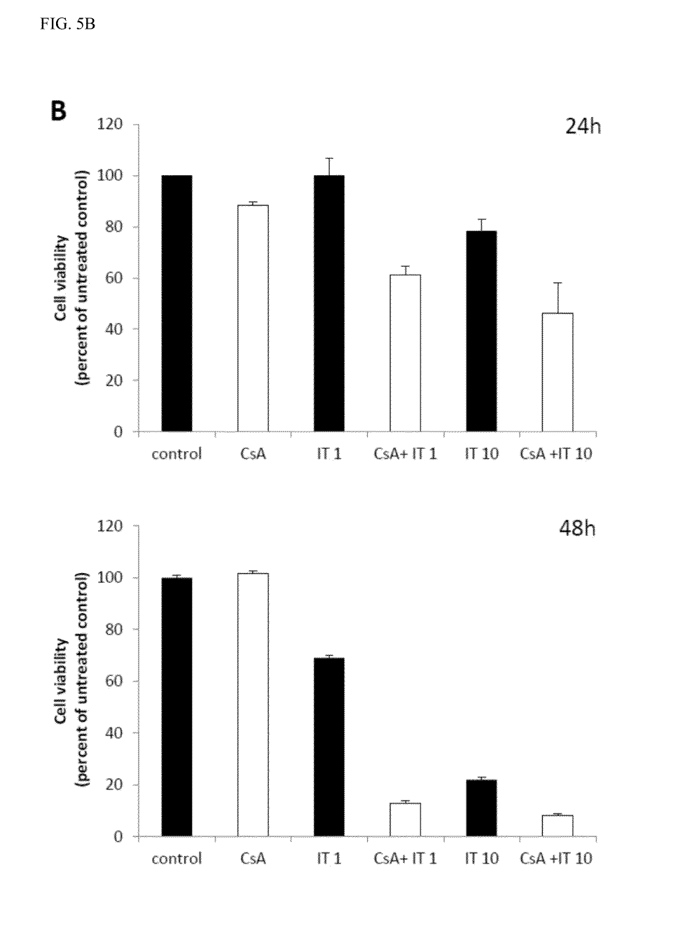

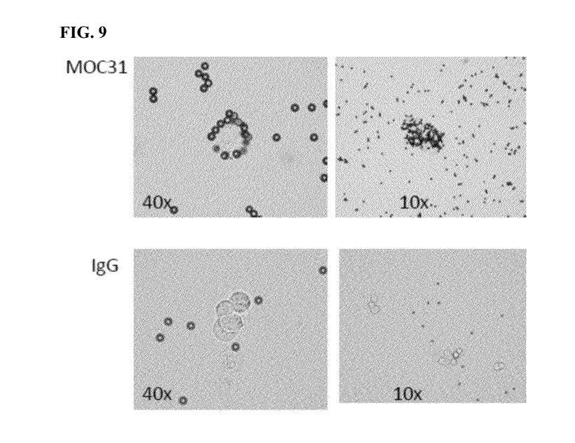

The ovarian cancer cell line B76 was used to investigate intracellular effects of MOC31PE (IT) and CsA on protein synthesis and cell viability. The expression of EpCAM is high in these cells (as assessed by immunomagnetic selection with MOC31 antibody-coated beads; FIG. 9). The ID₅₀ value for inhibition of protein synthesis was 8 ng/ml of MOC31PE (FIG. 5a). Cell viability was quantified in a MTS assay. In 10 ng/ml IT-treated cells the viability was decreased to 80 percent of untreated control (FIG. 5b). Protein synthesis was inhibited more efficiently when using the combination of IT with 2 μM CsA compared to IT treatment alone (FIG. 5a). By combining IT with CsA the ID₅₀ value for inhibition of protein synthesis with IT was ten times less than for IT alone. CsA alone showed none or negligible effects on protein synthesis and cytotoxicity. Although 1 ng/ml IT resulted in 20 percent reduction of protein synthesis, no significant reduction of cell viability was observed after 24 h (FIG. 5b). By extending the incubation period to 48 h, the fraction of metabolically active cells decreased further in all treatment groups (FIG. 5b). With 10 ng/ml IT alone 22 percent cell viability was observed, whereas the addition of CsA reduced the cell survival to only 13 percent.

MOC31PE Immunotoxin Induces Cell Membrane Damage and Reduces Cell Migration

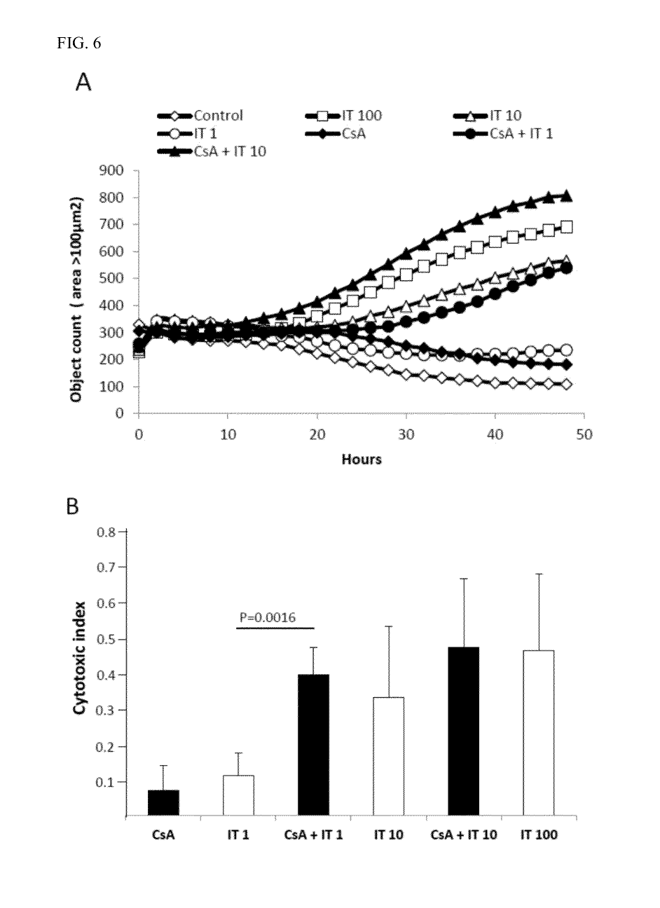

Membrane damage was determined by quantifying the number of fluorescent objects in an IncuCyte, where cells were analyzed every second hour for up to 48 h after adding the fluorescent probe YoYo-1. Addition of YoYo-1 alone did not induce membrane damage. No differences in the number of fluorescent objects were observed during the first 12 h of treatment, indicating intact cell membranes. The fluorescence increased in IT treated cells after approximately 15 h (FIG. 6a). FIG. 6b shows the cytotoxic index (CI) obtained after 48 h treatment. A dose dependent IT-response was observed with doses from 1 ng/ml to 100 ng/ml. The membranes of the cells were more damaged by the combination of IT and CsA, decreasing the IT dose needed by a factor of approximately ten compared to IT alone. Only a minor increase in CI was seen after exposure to CsA alone.

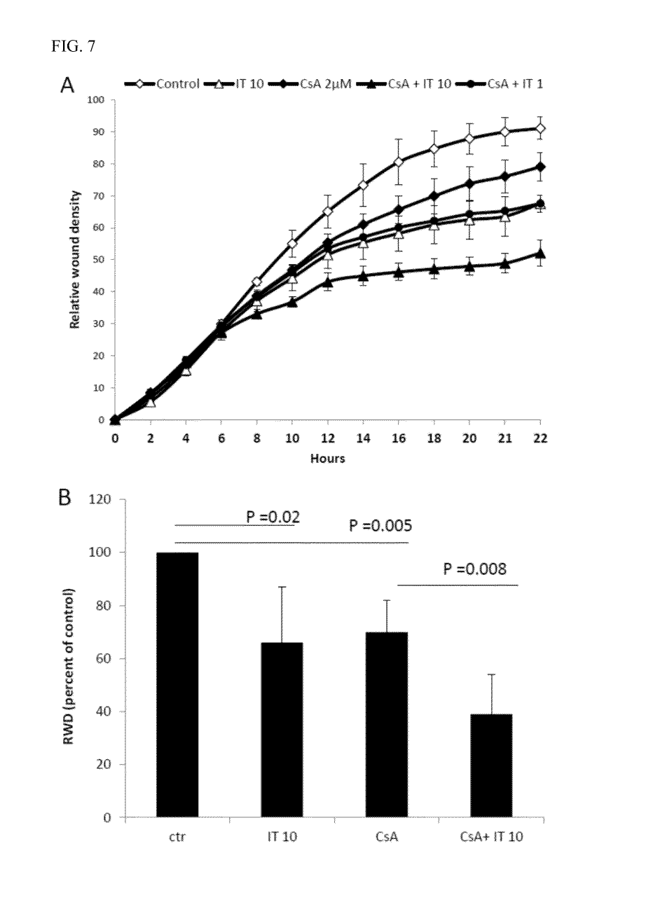

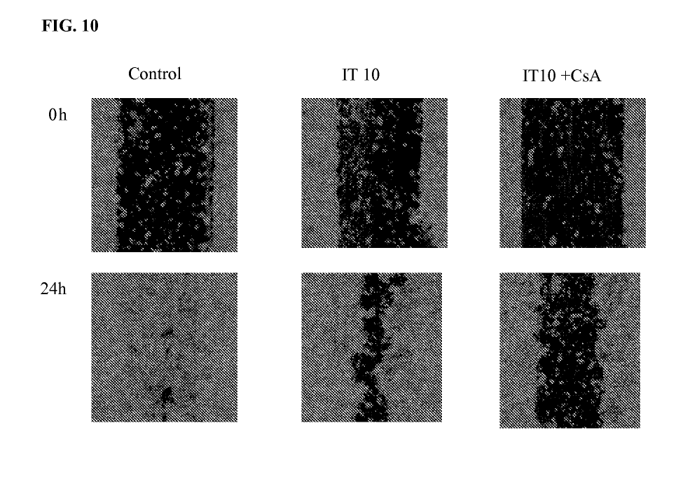

The wound healing assay mimics parts of the cancer metastasis process by measuring in vitro cell migration (Arwert et al., Nat Rev Cancer 2012, 12: 170-180). In control wells (untreated cells) the relative wound density (RWD) was 91 percent at start of the experiment (average of three wells, FIG. 7a) and pictures taken after 22 h revealed almost complete wound closure (FIG. 10). In wells containing cells treated with IT (10 ng/ml), cell migration was inhibited as the RWD decreased to 66 percent (p=0.02), and for CsA alone the RWD was 70 percent (p=0.005). A further reduction was observed when cells were treated with a combination of IT and CsA (RWD=39 percent, p=0.008). Results from five independent experiments are summarized in FIG. 7b.

Effects of MOC31PE Immunotoxin on Gene Expression

Previously, microarray analyses have revealed IT-induced differential expression of many transcripts (Risberg et al., J Immunotherapy 2011, 34: 438-447). To focus here on IT-induced changes in gene regulation two different PCR arrays were selected. One aim was to identify which cancer pathways were affected by IT treatment. The tumor metastasis array was used to study effects of the combination of CsA and IT, as this combination was previously shown to increase survival in a metastasis model in nude rats (Andersson et al., Br J Cancer 2009, 101: 1307-1315). In two independent experiments, mRNA was isolated from cells treated for 24 h with 10 ng/ml IT. Expression of 13 genes was more than two-fold changed in IT treated samples compared to non-treated controls (Table 2). Increased gene expression was detected for 11 targets and decreased expression for two targets. The Cq values in the control samples were 25 or more cycles for nine of the 13 affected gene products. Six of the detected gene products belong to the angiogenesis pathway. Moreover, increased mRNA levels were found for the transcription factors Jun, ETS2, and NFκβ1, which e.g. regulate the expression of tumor angiogenesis genes. The highest increase in expression was observed for THBS1 (Thrombospondin, 5.8 fold increase) and PDGFβ (platelet derived growth factor, 9 fold increase). These genes were selected for validation using qPCR with Taqman probes. RNA was isolated in a set of independent experiments from IT-treated samples and from non-treated controls. In six experiments median fold changed expression for IT treated samples compared to non-treated controls was 5.4 for PDGFβ (ranging from 2.1 to 31.1, p<0.02) and 10.5 for THBS1 (4.6 to 34.9, p<0.02). The fold change values for the specific mRNA transcript varied between experiments most likely due to high Cq values i.e. low expression of the mRNA. Within each experiment the variation between technical replicates was low, typically less than 0.5 cycles.

Using qPCR, possible effects of CsA alone and in combination with IT on expression of THBS1 and PDGFβ were also investigated. In CsA treated cells the expression of THBS1 and PDGFβ was two-fold reduced (n=2) compared to the expression in untreated control cells. In four independent experiments, the combination treatment compared to CsA alone treatment gave median fold changed expression of 34.5 (from 4.4 to 76.3, p<0.05) for THBS1 and of 13.9 for PDGFβ (4.5 to 41.3, p<0.05).

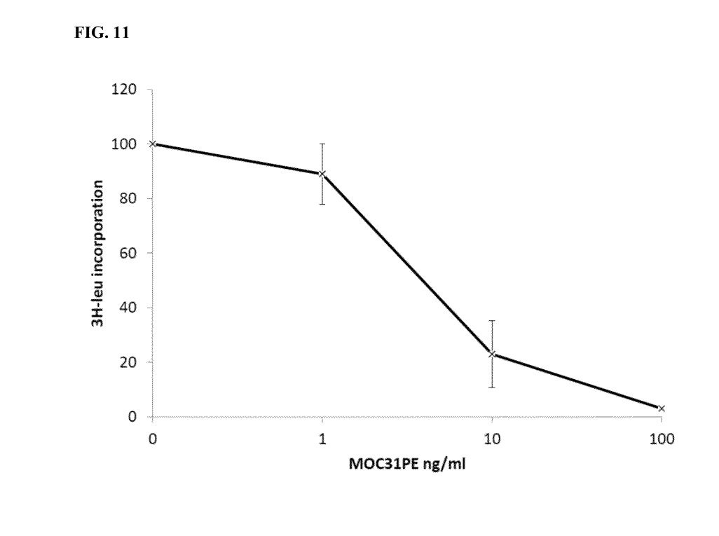

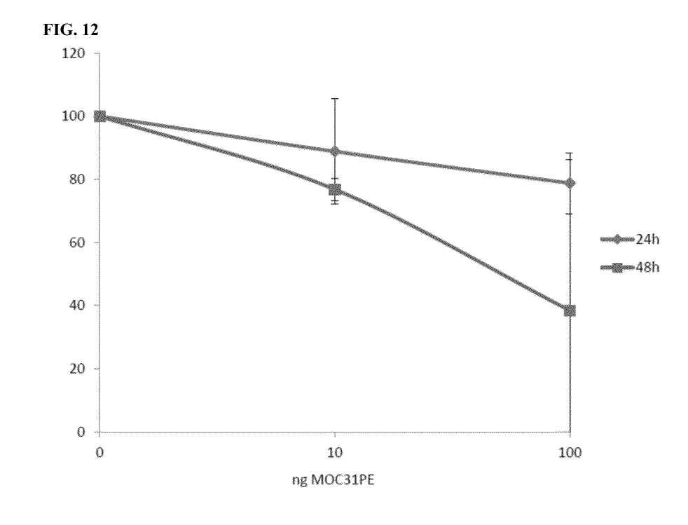

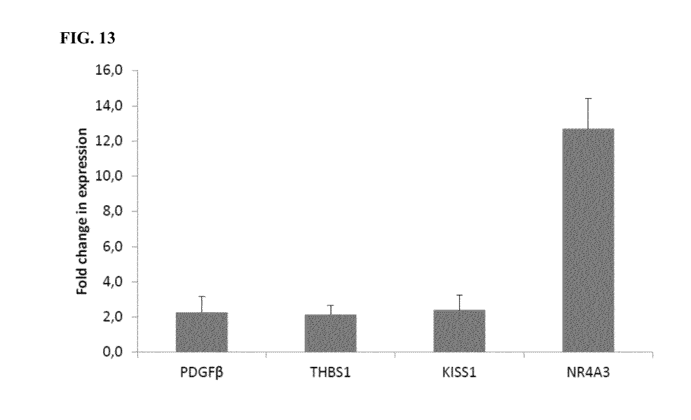

In the Tumor Metastasis Array, 23 of 84 gene products (Table 3) were found to be at least two-fold differentially expressed in the combination treatment compared to CsA alone treatment. Only one mRNA, coding for MYCL-1, was down regulated. The Cq-values for 16 of 23 mRNAs were 25 or higher in CsA-treated cells. Four gene products, coding for NR4A3 (nuclear receptor family 4 member 3), KISS1 (kisspeptin 1), NME4 (expressed in non-metastatic cells 4), and MMP9 (matrix metalloproteinase 9) were selected for validation using qPCR, and the results from the PCR array experiments were confirmed. The median fold changed expression was 16.4 for NR4A3 (ranging from 3 to 25.4, p<0.05), and 11.6 for KISS1 (ranging from 3 to 38.5, p<0.05) in four independent experiments. NME4 was up-regulated 3.8 fold and MMP9 only weakly up-regulated (2.6 times). KISS 1 and NR4A3 expression were increased also in cells treated with IT alone, confirming that the differential gene expression was independent of CsA. Expression of these transcripts was also analyzed after IT treatment of the ovarian cancer cell line HOC7. The inhibitory effect of MOC31PE on protein synthesis and decreased cell viability in HOC7 cells is shown in FIG. 11 and FIG. 12. The IT induced increase of NR4A3 expression was confirmed in this cell line (FIG. 13) and two-fold increase of THBS1, PDGFβ, and KISS1 transcripts were also detected.

Effects of MOC31PE Immunotoxin on NR4A3 Protein Expression and Subcellular Localization

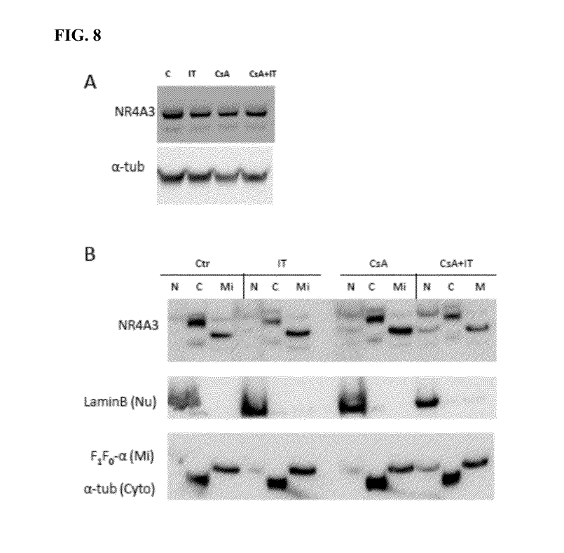

In the tested ovarian cancer cell lines, B76 and HOC7, treated with IT the largest increase in mRNA expression was observed for NR4A3. Immunoblot of B76 protein lysates with anti-NR4A3 antibody gave two proteins bands with apparent molecular weight of 55 and 60 kDa. No significant differences in protein level were seen when comparing the different treatments (FIG. 8a). Pro-survival as well as pro-apoptotic functions have been ascribed to NR4-family members. The pro-survival effect is due to transcription factor activity and localization in the nucleus, whereas the pro-apoptotic effect has been indicated to require mitochondrial localization. Subcellular fractionation was performed to identify the subcellular localization and possibly infer from this the mechanism for NR4A3 involvement during IT treatment. Three fractions enriched in either nuclear proteins, cytosolic proteins, or mitochondrial proteins were obtained and the purity of the different fractions was validated by immunoblotting (FIG. 8b). The cytosolic and nuclear fractions were acceptably pure (less than 10% contamination), whereas the mitochondrial marker protein was detected also in the nuclear fraction, especially when the cells had been treated with IT in combination with CsA. In this case the nuclear fraction contained 24 percent of the total F₁F₀-α ATP-synthase compared to 4 percent in the corresponding fraction from control cells. In fractions from control cells NR4A3 was detected mainly as a 60 kDa band in the cytosol and as a 55 kDa band in the mitochondrial fraction. A faint band around 60 kDa was detected in the nuclear fraction, but in this fraction a 100 kDa band was also observed. IT treatment increased the amount of NR4A3 in the mitochondrial fraction, indicating a pro-apoptotic function of NR4A3 (FIG. 8b). This increase was also observed in mitochondrial fraction from CsA treated cells, and was accompanied by increased NR4A3 in the nuclear fraction, indicating increased transcription of NR4A3 regulated genes. NR4A3 was reduced in the mitochondrial fraction from IT+CsA treated cells and further increased in the nuclear fraction. Increased amount of the two NR4A3 bands and detection of the mitochondrial marker in the nuclear fraction indicates altered intracellular compartment for mitochondrial proteins as could be expected in cells undergoing apoptosis.

The major limitation to curative therapy for ovarian cancer is acquired drug resistance to the chemotherapeutic agents used, such as Carboplatin and Paclitaxel. An additional drawback is the induced severe side-effects, mainly caused by the non-cancer cell specificity of the agents, reducing the patients' quality of life. It is therefore useful to identify novel drugs, which circumvent these disadvantages for successful treatment of ovarian cancer. In the present study, it was demonstrated in several different assays that the MOC31PE effectively inhibits protein synthesis, proliferation and cell survival of ovarian cancer cells, B76 and HOC7.

The MOC31PE only binds to and kill cells expressing the antigen EpCAM, which is expressed in more than 90% of all epithelial ovarian carcinomas and to a negligible amount on normal cells, reducing the possibility of IT induced side effects in patients.

Gene expression analysis was performed to identify affected signaling pathways induced by the treatments and several interesting candidate genes were found. In the Cancer Pathway Finder array, the majority of the genes affected by MOC31PE were related to angiogenesis, reflecting the importance of this cancer pathway in B76 cell growth. The two genes with the highest fold increase in expression on the array, PDGFβ and THBS1, was confirmed by qPCR. The PDGF network was recently identified as a biomarker for prognosis in ovarian cancer where higher levels of PDGF pathway activity were associated with reduced survival (Ben-Hamo et al., BMC Systems Biology 2012, 6:3). The angiogenesis inhibitor Bevacizumab (Avastin), that binds to VEGF A, is an used molecular target agent in ovarian cancer (Itamochi H: World Journal of Biological Chemistry 2010, 1: 209-2200). Given the importance of the PDGF pathway, targeting of VEGF, PDGF, and FGF at the same time may be more effective than targeting only VEGF (Bell-McGuinn K, Konner J, Tew W, Spriggs DR: New drugs for ovarian cancer. Annals of Oncology 2011, 22: viii77-viii82). THBS1 was the first endogenous angiogenesis inhibitor identified (Good et al., Proceedings of the National Academy of Sciences 1990, 87: 6624-6628). A role in cancer progression and cancer inhibition has been ascribed to the protein, and different effects of THBS1 depending on the phase of the progression have been suggested (Kazerounian et al., Cellular and Molecular Life Science 2008, 65: 700-712). In an early stage, high stromal expression of THBS1 inhibits tumor growth whereas later in the vascularized tumor THBS1 may increase the adhesive properties of tumor cells or modulate extracellular matrix proteins thereby promoting tumor invasion. It was observed that CsA mono-treatment inhibited migration and reduced expression of some transcripts, including THBS1 in addition to potentiating IT effects. Calcineurin, the phosphatase inhibited by CsA, has been reported to regulate transcription of CTSK (Combs et al., Development 2011, 138: 1747-1757) and CXCR4 (Cristillo et al., Molecular Immunology 2003, 40: 539-553); two of five other affected genes. The inhibition of B76 cell migration by IT+CsA treatment may be a result of reduced THBS1 and/or MMP9 protein levels since increased transcription cannot be accompanied by increased translation due to IT-induced protein synthesis inhibition. In the tumor metastasis array mainly increased gene expression was seen when comparing CsA alone versus CsA+MOC31PE treatment of B76 cells. Examples of genes influenced are the metastasis suppressor KISS1 and its receptor. In ovarian carcinoma the increased expression of KISS1 has been shown to inhibit cell migration (Hata et al., European Journal of Cancer 2007, 43: 1452-1459). This supports the results from the scratch-wound healing assay showing decreased migration in the B76 cells treated with MOC31PE alone or MOC31PE+CsA. Higher expression of KISS1 may also sensitize cancer cells for chemotherapy (Jiffar et al., Oncogene 2011, 30: 3163-3173). Thus, the results support a contribution of MOC31PE as a supplement to reduce chemotherapy resistance in ovarian cancer treatment.

The largest up-regulation was observed for the nuclear hormone receptor NR4A3, a member of the NR4A subfamily with poorly understood biological function and unknown physiological ligands (Pearen et al., Molecular Endocrinology 2010, 24: 1891-1903.). Depending on the context, NR4A transcription factors may be pro-survival factors or induce cell death (Moll et al., Oncogene 2006, 25: 4725-4723). Knock-out mice without NR4A3 (Nor-1) and NR4A1 (Nur77) developed spontaneous acute myeloid leukemia (Mullican et al., Nature Medicine 2007, 13: 730-735), indicating tumor suppressing effects. In cancer cells, growth factors and mitogens induce expression of these transcription factors, indicating a role in cancer growth (Moll et al., supra). However, induction of NR4A1 also occurs in response to apoptosis inducing factors in cancer cells. When translocated to mitochondria NR4A1 binds BCL-2, thereby inducing apoptotic cell death (Moll et al., supra) and during apoptosis in thymocytes mitochondrial targeting of NR4A3 was observed (Thompson et al., Eur J Immunol 2010, 40: 2041-2049). In B76 cells, the majority of the NR4A3 protein was located in the cytosol. Two main changes in intracellular distribution were observed. MOC31PE or CsA shifted the protein to the mitochondrial fraction compatible with induction of apoptosis. Especially in MOC31PE+CsA treated cells increased NR4A3 was detected in the nuclear fraction. Increased amount of 60 kDa protein points to increased transcription of its target genes. Since increased 55 kDa protein in the nuclear fraction was accompanied by increased mitochondrial marker protein, and the nuclear fraction was pelleted at low speed, this implies that the mitochondrial mass has increased or that mitochondria have fused to larger structures. This is most likely an effect of the ongoing cell death. The increase in NR4A3 transcript, signals a need for NR4A3 protein synthesis. No corresponding increased NR4A3 protein was detected as IT inhibits protein synthesis, but translocation of NR4A3 to mitochondria enriched fractions indicates a role for this protein in MOC3JPE-induced cell-death.

In summary, these results show that a PE-containing IT, MOC31PE, induces transcription of mRNAs for genes involved in angiogenesis and tumor metastasis. In addition, the therapeutic use of MOC31PE alone or in combination with CsA provides an approach to the treatment of recurrent/chemoresistant ovarian carcinoma.

TABLE 2
Fold change in gene expression comparing control (untreated
cells) and 10 ng/ml IT treated B76 cells. Results from
two independent experiments are analysed together.

| | | | Fold |
|---|---|---|---|
| Cancer pathway | Gene | Description | Change |
| Adhesion | ITGα3 | Integrin α3 | −2.1 |
| Angiogenesis | FGFR2 | Fibroblast growth factor | 2.1 |
| | | receptor 2 | |
| | IFNβ1 | Interferon β | 3.8 |
| | TNF | Tumor necrosis factor | 3.8 |
| | IL8 | Interleukin 8 | 5.0 |
| | THBS1 | Thrombospondin | 5.8 |
| | PDGFβ | Platelet derived growth | 9.0 |
| | | factor β | |
| Cell cycle control | CDC25A | CDC25 phosphatase family | 2.4 |
| and DNA damage | CDKN1A | Cyclin dependent kinase | 2.4 |
| repair | | inhibitor | |
| Signal transduction | ERBB2 | Epidermal growth factor | −2.9 |
| molecules and tran- | | receptor family | |
| scription factors | ETS2 | Transcription factor | 3.2 |
| | NFκβ1 | Transcription factor | 3.2 |
| | JUN | Transcription factor | 5.4 |

TABLE 3
Fold change in gene expression comparing CsA treated B76 cells with or without
10 ng/ml IT. Results from two independent experiments are analysed together.

| Cancer | | | Fold |
|---|---|---|---|
| pathway | Gene | Description | Change |
| Cell adhesion | PNN | Pinin, desmosome-associated protein | 2.2 |
| | FAT1 | Cadherin-related tumor suppressor homolog | 2.3 |
| Cell cyclus | BRMS1 | Breast cancer metastase suppressor, transcriptional repressor | 2.0 |
| | NF2 | Neurofibromin | 2.2 |
| Cell cyclus and | RB1 | Retinoblastoma, tumor suppressor, transcriptional repressor | 2.2 |
| transcription factor | TP53 | Tumor suppressor, transcription factor | 2.3 |
| Cell cyclus or cell | NME1 | Expressed in Non-Metastatic cells, nucleoside diphosphate kinase | 3.2 |
| proliferation | | | |
| Cell growth and | SSTR2 | Somatostatin receptor 2, ligand somatostatin 14/28 | 2.1 |
| proliferation | DENR | Density-regulated protein, involved in translation | 2.6 |
| | KISS1R | Receptor for KISS1 | 2.8 |
| | FLT4 | Receptor tyrosine kinase, ligand VEGF C/D | 3.1 |
| | CXCR4 | CXC chemokine receptor, ligand SDF-1 | 3.4 |
| | EPHB2 | Receptor tyrosine kinase, ligand ephrin-family members | 10.3 |
| Invasion | MMP10 | Matrix metalloproteinase | 2.4 |
| | MMP9 | Matrix metalloproteinase | 5.3 |
| Other | METAP2 | Methionyl aminopeptidase | 2.1 |
| | CD82 | Metastasis suppressor | 2.2 |
| | CTSK | Cathepsin K, cysteine protease | 4.2 |
| | NME4 | Expressed in Non-Metastatic cells, nucleoside diphosphate kinase | 7.6 |
| | KISS1 | Metastasis suppressor | 14.6 |
| Transcription | MYCL1 | Myc-related | −3.8 |
| factor | SMAD4 | SMAD family member, | 2.0 |
| | NR4A3 | Nuclear-receptor subfamily 4 member A3, potential transcriptional activator | 20.6 |

TABLE 4
Taqman probe/primers from Applied Biosystems (Life Technology)
that were used for validation gene-expression data that
were observed with the PCR array technology.

| Gene | Taqman probe/primer (Applied) |
|---|---|
| THBS-1 | Hs00962908 m1 |
| PDGF β | Hs00966522 m1 |
| KISS1 | Hs00158486 m1 |
| NR4A3 | Hs00545007 m1 |
| NME4 | Hs00359037 m1 |
| MMP9 | Hs00234579 m1 |
| RPL37a | Hs01102345 m1 |

### Example 3

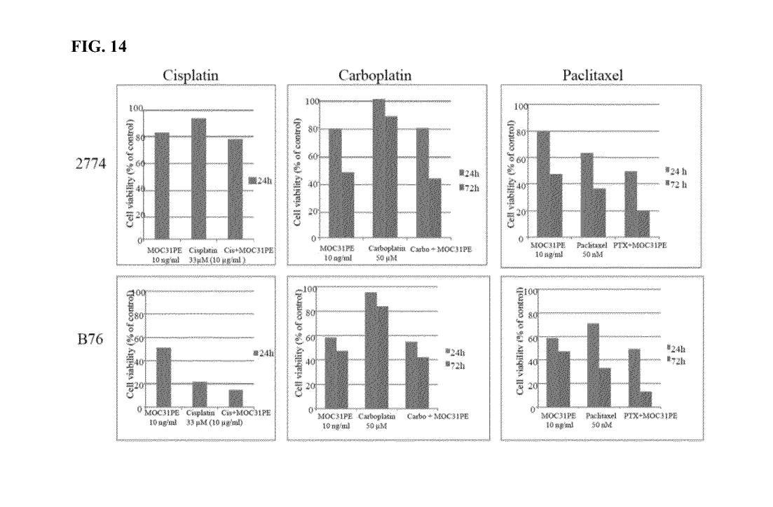

It was investigated whether MOC31PE alone or in combination with the most common chemotherapies for ovarian cancer could affect ovarian cancer cell viability (FIG. 14). The ovarian cancer cell lines B76 and 2774 were treated for 24 or 72 h with MOC31PE and/or cisplatin/carboplatin/paclitaxel and cell viability was measured using the CellTiter 96® Aqueous One Solution (MTS-assay) (Promega, Madison, Wis.). The values for viability of the treated cells were compared to the values generated for the untreated control cells and reported as the percentage of cell viability. The assays were performed in triplicate, and repeated at least three times. MOC31PE efficiently decreased cell viability in a dose-dependent manner (data not shown). The inhibitory dose 50 (ID₅₀) of MOC31PE was approx 10 ng/ml for 72 h in both cell lines. MOC31PE in combination with paclitaxel induced additive cytotoxicity in both ovarian cancer cells, whereas the two other drugs in combination with MOC31PE did not additive effect. Importantly, no antagonistic effect was observed indicating that MOC31PE could be used in clinical setting in combination with standard drugs for ovarian cancer treatment.

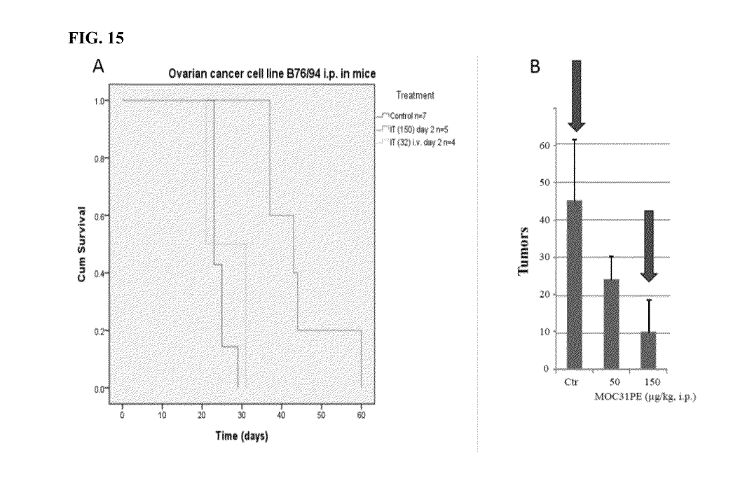

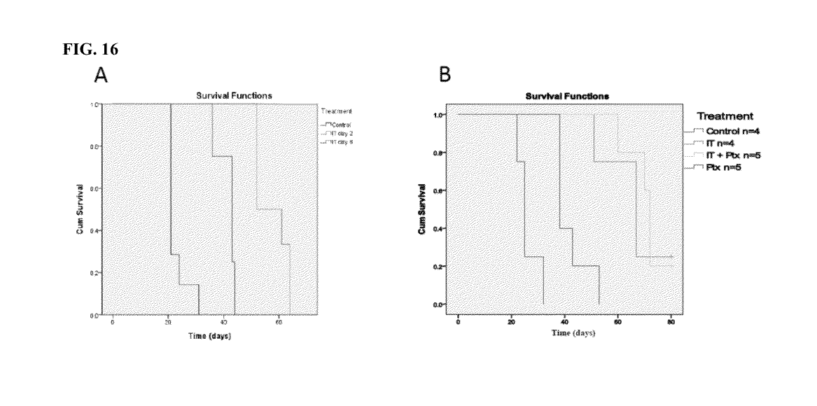

The therapeutic potential of intraperitoneal administration of MOC31PE was then investigated in two experimental mouse models based on the ovarian cancer cell lines B76 (FIG. 15) and 2774 (FIG. 16). The animals were divided into three groups. Day 1 the animals were intraperitoneal injected with B76 cancer cell to mimic the situation in patients after cytoreductive surgery, were non-visible cancer cells are still left. MOC31PE treatment was initiated the day after (Day 2) or on day 8, as indicated by giving either MOC31PE 50 μg/kg intraperitoneal injection (i.p.) or 32 μg/kg intravenous injection (i.v.). No adverse events were recorded. Disease progression, as increase of abdominal size and other signs of symptoms, were measured twice weekly. Body weight was recorded throughout the experiments. Survival as a function of treatment was analysed by Kaplan-Meier plots.

Intraperitoneal administration of MOC31PE strongly inhibited formation of peritoneal carcinomatosis in the B76 model (FIG. 15A). A statistically significant prolongation in survival (P<0.005) with MOC31PE (i.p.) compared to control animals was obtained, whereas MOC31PE 32 μg/kg i.v. had no effect. A dose-dependent manner decrease in numbers of tumorlesions in this animal model was also demonstrated (FIG. 15B).

Similar therapeutic effects of i.p. administration of MOC31PE were also demonstrated in the 2774 ovarian cancer model (FIG. 16). MOC31PE administered on day 2 strongly inhibited formation of peritoneal carcinomatosis, whereas MOC31PE administered on day 8 showed reduced, but still substantial, improvement in survival compared to control animals (FIG. 16A). The effect of combinatorial treatment was subsequently investigated (FIG. 16B). MOC31PE treatment was initiated on day 2, either alone or in combination with Paclitaxel (Ptx), while one group was given Ptx alone. Survival was compared to that of control animals (blue line, n=4). Ptx treatment alone has a significant effect on disease progression, however MOC31PE show a superior effect compared to Ptx alone. The combination therapy shown a trend towards improved cytotoxic effect, but this did not reach the level of statistical significance Importantly, the combination of PTX and MOC31PE showed no antagonist effect, support the clinical application of the combination.

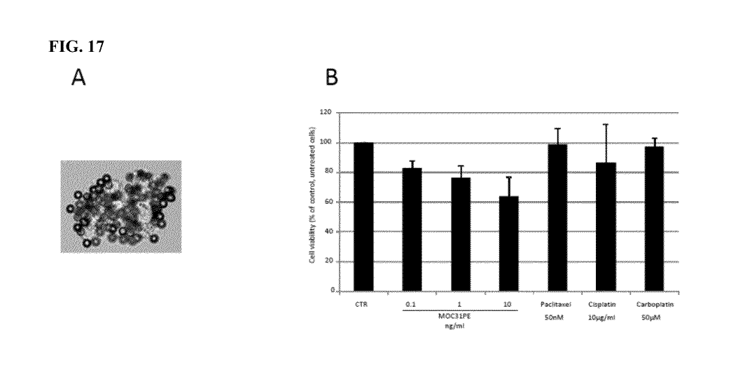

The effect of MOC31PE was further analysed ex vivo in short-term cultures of fresh human ovarian tumor tissue taken directly from the operating theatre (FIG. 17). The ovarian tumor tissue was disaggregated, the cancer cells counted, and a portion of the cells analyzed for expression of EpCAM-positive ovarian cancer cells by using immunomagnetic MOC31-coated beads. FIG. 17A, shows EpCAM-positive ovarian cancer cells. As shown, the ovarian cancer cells are clearly EpCAM-positive, which is the antigen target for MOC31PE.

The rest of the ovarian cancer cells were seeded directly into 96-wells plate for cell viability assay and seeded into cell culture flasks, to establish a cell culture. After 10-12 days culture, the cells were treated with MOC31PE and/or cisplatin/carboplatin/paclitaxel and analysed for viability (FIG. 17B). The cells were incubated at 37° C. for 24 h. Thereafter the CellTiter 96 Aqueous One Solution was added to the wells, and the absorbance was measured 4 h later at a wavelength of 490 nm. The values for viability of the treated cells were compared to the values generated for the untreated control cells and reported as the percentage of cell viability. The assays were performed in triplicate. Error bars indicate standard deviation from the three experiments. MOC31PE induces a dose-dependent decrease in cell viability, while the anti-cancer drugs showed minimal effect. These result further support the use of MOC31PE for treatment of ovarian cancer.

We claim:

1. A method of treating a cancer that expresses EPCAM, comprising:
administering an immunotoxin comprising and antibody that binds to EpCAM to a subject diagnosed with a cancer that expresses EpCAM.

2. The method of claim 1, wherein said immunotoxin comprises a cytotoxic agent conjugated to said antibody.

3. The method of claim 1, wherein said cancer is a mucinous peritoneal surface malignancy or peritoneal carcinomatosis.

4. The method of claim 1, wherein said administering is via intraperitoneal injection.

5. The method of claim 1, wherein said method further comprises the step of administering an additional anticancer agent.

6. The method of claim 5, wherein said agent is mitomycin C.

7. The method of claim 1, wherein said administering reduces symptoms, growth, or presence of said epithelial cancer.

8. The method of claim 1, wherein said subject has undergone cytoreductive surgery.

9. The method of claim 1, wherein said immunotoxin is MOC31PE.

10. The method of claim 9, wherein said MOC31PE antibody is administered at a dose of 5 to 150 μg/kg.

11. The method of claim 6, wherein said mitomycin C is administered at a dose of 1 to 5 mg/kg.

12. The method of claim 6, wherein said immunotoxin and said chemotherapeutic agent are administered concurrently or successively.

13. The method of claim 6, wherein at least one of said immunotoxin and said chemotherapeutic agent are administered intraperitoneally.

14. A method of treating a cancer that expresses EPCAM, comprising:
a) assaying a sample from a subject diagnosed with cancer for the presence of EpCAM expression; and
b) administering an immunotoxin comprising an antibody that binds to EpCAM when said cancer expresses EpCAM.

15. The method of claim 14, wherein said immunotoxin comprises a cytotoxic agent conjugated to said antibody.

16. The method of claim 14, wherein said cancer is a mucinous peritoneal surface malignancy or peritoneal carcinomatosis.

17. The method of claim 14, wherein said administering is via intraperitoneal injection.

18. The method of claim 14, wherein said method further comprises the step of administering an additional anticancer agent.

19. The method of claim 18, wherein said agent is mitomycin C.
